(19) United States
(12) Patent Application Publication
(10) Pub. No.: US 2026/0226020 A1
(43) Pub. Date: Aug. 6, 2026

(54) Nav 1.7 AND/OR Nav 1.8 BLOCKING HYDRAZIDES, PROCESSES FOR THE PREPARATION THEREOF, COMPOSITIONS, USES, METHODS OF TREATMENT USING SAME AND KITS

(71) Applicants: Eurofarma Laboratórios S.A., São Paulo (BR); Universidade Federal do Rio de Janeiro - UFRJ, Rio de Janeiro (BR)

(72) Inventors: GABRIELA BARREIRO, São Paulo (BR); DANILO PEREIRA DE SANT'ANA, São Paulo (BR); JÚLIA LAMMOGLIA MONTEIRO, São Paulo (BR); LUIS EDUARDO REINA GAMBA, São Paulo (BR); LÍDIA MOREIRA LIMA, Rio de Janeiro (BR); CARLOS ALBERTO MANSSOUR FRAGA, Rio de Janeiro (BR); ELIEZER JESUS DE LACERDA BARREIRO, Rio de Janeiro (BR)

(21) Appl. No.: 19/152,110

(22) PCT Filed: Jan. 26, 2024
(86) PCT No.: PCT/BR2024/050024

§ 371 Date: Jul. 30, 2025

Related U.S. Application Data
(60) Provisional application No. 63/482,235, filed on Jan. 30, 2023.

(51) Int. Cl. C07D 401/04; A61K 31/4418; A61K 31/497; A61K 31/50; A61K 31/505; A61K 31/553; C07D 213/87; C07D 237/24; C07D 239/28; C07D 241/24; C07D 401/12; C07D 401/14; C07D 403/12; C07D 413/12

(52) U.S. Cl. CPC C07D 401/04; A61K 31/4418; A61K 31/497; A61K 31/50; A61K 31/505; A61K 31/553; C07D 213/87; C07D 237/24; C07D 239/28; C07D 241/24; C07D 401/12; C07D 401/14; C07D 403/12; C07D 413/12

(57) ABSTRACT

The present invention refers to Nav 1.7 and/or Nav 1.8 blocking hydrazides. More specifically, the present invention is related to hydrazides comprising Formula (I), in which the substituents R₁ to R₈ are selected independently of the groups defined in the specification, as well as their processes of obtaining, compositions comprising at least one of these compounds, uses, treatment methods to treat or prevent pain-related pathologies and kits. The present invention belongs to the fields of medicinal chemistry, organic synthesis, as well as in treatment of pain-related diseases.

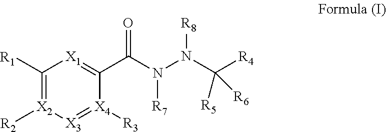

### FIELD OF INVENTION

The present invention refers to Nav 1.7 and/or Nav 1.8 blocking hydrazides, their obtaining processes, compositions containing same, uses, kits and treatment methods to treat or prevent pain-related pathologies. The present invention belongs to the fields of medicinal chemistry, organic synthesis, as well as in treatment of pain-related diseases.

### BACKGROUND OF INVENTION

Physiological pain is an important protective mechanism designed to alert the body to real or potential injuries that can put its integrity at risk. Broadly speaking, physiological pain can be classified into nociceptive pain and inflammatory pain. Nociceptive pain is characterized by having a high activation threshold, which remains until the stimulus that generated it is eliminated. Inflammatory pain, which arises as a response to tissue damage, is characterized by having a low activation threshold and is a consequence of the activity of molecular mediators of the inflammatory process in sensitizing nociceptors (Schaible. Langenbecks Arch. Surg. 2004, 389, 237). When these nociceptive processes remain in the absence of noxious stimuli or in response to non noxious, stimuli, the protective and repair role of pain loses its functionality, configuring a maladaptive picture of neural plasticity and, as a consequence, a pathological state of chronic pain. Among the syndromes included in this classification, neuropathic pain has a high prevalence and impact today (Smith. Pain. 2020, 161, 1:S127; Cavalli. Int. J. Immunopathol. Pharmacol. 2019, 33:2058738419838383; Bouhassira. Rev Neurol (Paris). 2019, 175(1-2):16; Scholz. Nature Neurosci., 2002, 5,1062; Costigan. Annu. Rev. Neurosci., 2009, 32, 1).

Neuropathic pain is defined by the International Association for the Study of Pain (IASP) as pain initiated or caused by a primary dysfunction or injury in the central and/or peripheral nervous system (Dworkin. Clin. J. Pain, 2002, 18(6), 343). Central neuropathic pain comes from spinal cord injuries or central nervous system diseases such as multiple sclerosis or Parkinson's disease (Ducreux. Brain, 2006, 129, 963). Peripheral neuropathic pain, on the other hand, can be caused by trauma, metabolic disorders, chemical neurotoxicity, infection, or tumor invasion, among others. Among the most common syndromes of neuropathic pain are chemotherapy-induced neuropathic pain, complex regional pain, neuropathy related to viral infection, neuropathy secondary to tumor infiltration, diabetic neuropathy, phantom limb pain, postherpetic neuralgia, trigeminal neuralgia, and postsurgical neuralgia (Pak. Curr. Pain Headache Rep., 2018, 22(2), 9).

Currently, there is no specific treatment for the control of pathologies related to neuropathic pain, however, the first-line alternative consists of the use of opioid analgesics, and—as adjuvants—local anesthetics, anticonvulsants and antidepressants. However, the adverse effects and low efficacy drastically limit the use of these agents in the control of various pain-related pathologies (Kushnarev. Expert Opin. Investig. Drugs, 2020, 29(3), 259; Emery. Expert Opin. Ther. Targets, 2016, 20(8), 975).

Voltage-gated sodium channels (Nav) play a key role in the transmission of pain-related stimuli. These channels are activated in response to membrane depolarization, allowing the generation and propagation of action potentials in neurons (and other electrically excitable cells), by controlling the flow of sodium ions through the membranes. Structurally, voltage-gated sodium channels are heteromeric transmembrane proteins consisting of one a subunit and two β auxiliary subunits. The α subunit is organized into four homologous domains (I-IV), each with six transmembrane segments (S1-S6). The S4 segment of each domain is characterized by presenting a conserved region of arginine residues, which act as sensors of the intra- and extracellular electrical environment of the neuron. This mechanism makes it possible to transform changes in the cellular electric field into specific conformational changes that, in turn, regulate the activation, deactivation and inactivation of voltage-gated sodium channels (Catterall. Nat. Chem. Biol., 2020, 16, 1314; Wisedchaisri. Cell., 2019, 178(4), 993; Clairfeuille. Science, 2019, 363, 1302).

In mammals, nine subunits α (Nav 1.1-Nav 1.9) and four auxiliary subunits β (β31-β34) have been identified. The α subunits can further be classified according to their susceptibility to blocking by tetrodotoxin (TTX), being classified as sensitive to tetrodotoxin (Nav 1.1, Nav 1.2, Nav 1.3, Nav 1.4, Nav 1.6 and Nav 1.7) or resistant to tetrodotoxin (Nav 1.5, Nav 1.8 and Nav 1.9) (Lera-Ruiz. J. Med. Chem., 2015, 58 (18), 7093; Bagal. J. Med. Chem., 2013, 56(3), 593). Each of these subunits α has a different profile of expression and function, so that some of them are essential for the proper functioning of organs such as the heart and/or brain. Thus, the non-selective blockade of these channels is related to several types of adverse effects, such as migraine, epilepsy, paralysis and muscle and cardiac syndromes, among others (Bagal. J. Med. Chem., 2013, 56(3), 593; Bagal. Channels, 2015, 9(6), 360).

Broadly speaking, sodium channels are distributed mainly in the central and peripheral nervous system, in neurons and glia. Nav channels 1.1, 1.2, and 1.3 are primarily expressed in the brain. The Nav 1.4 and Nav 1.5 channels are found primarily in skeletal and cardiac muscles, respectively. Nav 1.6 channels are expressed in the central and peripheral nervous systems, while Nav 1.9 channels are selectively expressed in C-type nociceptive fibers in the dorsal root ganglion. On the other hand, the Nav 1.7 and Nav 1.8 channels are mainly found in the peripheral nervous system and are directly related to the processes of pain transmission (Law. Drug Discovery Today, 2019, 24(7), 1389; Bagal. Channels (Austin), 2015, 9(6), 360; Lera-Ruiz. J. Med. Chem., 2015, 58 (18), 7093).

Nav 1.7 sodium channels are expressed broadly in the olfactory epithelium, sympathetic ganglion, and dorsal root ganglion, predominantly in nociceptive fibers C and A5. A large amount of evidence supports the important role of Nav 1.7 sodium channels in pain transmission processes. For example, gain-of-function related mutations in the gene (SCN9A), which encodes sodium channel Nav 1.7, are associated with extreme pain disorders such as congenital pain insensitivity, paroxysmal extreme pain disorder, and primary erythromelalgia. On the other hand, mutations related to loss of gene function (SCN9A) are related to congenital insensitivity to pain in individuals who, in general terms, are free of motor or cognitive impairment (Vetter. Pharmacology & Therapeutics, 2017, 172, 73; Ahuja. Science, 2015, 350(6267), 1491; Kingwell. Nat. Rev. Drug Discov., 2019, 18, 321; Safina. J. Med. Chem., 2021, 64, 2953; Luo. J. Med. Chem., 2019, 62, 831; Bankar. Cell Reports, 2018, 24, 3133).

Nav 1.8 sodium channels are most expressed in the peripheral nervous system, widely (but not exclusively) in C-type nociceptive fibers in the dorsal root ganglion. Recent evidence including elevated expression levels of Nav 1.8 in chronic pain states, data with Nav 1.8 knockout animals, and analgesic activity of Nav 1.8-specific desensitizing oligodeoxynucleotides, among others (Brown. Bioorg. Med. Chem., 2019, 27(1), 230; Payne. Br. J. Pharmacol., 2015, 172(10), 2654; Bagal. Med. Chem. Lett. 2015, 6(6) 650; Kort. J. Med. Chem. 2008, 51, 407; Zhang. Neuropharmacology, 2010, 59, 201 and 207), support the role of the sodium channel Nav 1.8 in the development and process of pain-related pathologies (Kingwell. Nat. Rev. Drug Discov., 2019, 18, 321; Law. Drug Discovery Today, 2019, 24(7), 1389; Bagal. Channels (Austin), 2015, 9(6), 360; Lera-Ruiz. J. Med. Chem., 2015, 58(18), 7093).

Thus, the voltage-gated sodium channels Nav 1.7 and Nav 1.8 are considered promising therapeutic targets for the treatment of neuropathic pain-related dysfunctions (Kornecook. J. Pharmacol. Exp. Ther., 2017, 362, 146; Kingwell. Nat. Rev. Drug Discov., 2019, 18, 321; Bagal. Channels (Austin), 2015, 9(6), 360; Lera-Ruiz. J. Med. Chem., 2015, 58(18), 7093; Deuis. Neuropharmacology, 2017, 127, 87 and 108; Kushnarev. Expert Opin. Investig. Drugs, 2020, 29(3), 259; McKerrall. Bioorganic & Medicinal Chemistry Lett., 2018, 28, 3141; Emery. Expert Opin. Ther. Targets, 2016, 20(8), 975; Bagal. Bioorganic & Medicinal Chemistry Lett., 2014, 24, 3690).

A large number of compounds have been described in the literature for their ability to act as blockers of Nav 1.7 and 1.8 sodium channels, however, they present a great structural diversity, a fact that does not allow the establishment of a common pharmacophoric group.

The patent literature contains several examples of compounds that act as sodium channel blockers. In particular, Nav 1.7 selective sodium channel blockers are described in U.S. Ser. No. 10/550,080, U.S. Pat. No. 9,765,029, and U.S. Ser. No. 10/000,475. Additionally, some documents describe selective blockers of Nav 1.8 sodium channels as WO2020261114, WO2020092667, U.S. Pat. No. 9,163,042, WO2014120808, WO2014120815, WO2018213426, WO2019014352, WO2015006280, and U.S. Pat. No. 7,928,107. These documents reveal compounds with different structures from the present invention.

In addition, there are patent documents that describe dual Nav blockers 1.7 and 1.8, including WO2018235851, U.S. Pat. No. 8,629,149, JP2017001991 that reveal, respectively, pyridyl amines, oxopiperazine derivatives and benzoxazolons. However, all these documents reveal compounds with structures and physicochemical characteristics different from the present invention.

In this context, it is advantageous to develop new alternatives of compounds that can act as Nav 1.7 and/or Nav 1.8 blockers that have adequate pharmacological action and, preferably, provide mitigated adverse effects. Therefore, the present invention refers to hydrazides as an alternative and/or complement to the treatment of pain-related diseases.

### SUMMARY OF THE INVENTION

The present invention discloses hydrazides with blocking activity of Nav 1.7 and/or 1.8 channels, against pain-related pathologies, as well as compositions, uses, kits, treatment methods and related preparation processes.

The present invention refers to compound(s) of Formula (I):

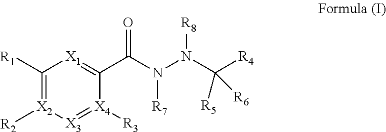

or a pharmaceutically acceptable salt, hydrate, solvate, ester and isomer thereof, wherein:
   X₁, X₂, X₃, and X₄ are independently selected from the group consisting of carbon, CH, or nitrogen, where:
   when at least one of the substituents X₂, X₄ is N or CH, the corresponding R (R₂, R₃) is null.
   R₁ is:

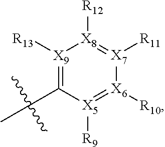

where:
X₅, X₆, X₇, X₈, and X₉ are independently selected from the group consisting of carbon, CH, or nitrogen, where:
when at least one of the substituents X₅-X₉ is N or CH, the corresponding R (R₉—R₁₃) is null;
R₉, R₁₀, R₁₁, R₁₂, and R₁₃ are independently selected from the group consisting of hydrogen, halogen, linear or branched C₁-C₆ alkyl, linear or branched C₁-C₆ alkoxy, cycloalkoxy C₃-C₇, difluoromethyl, trifluoromethyl, linear or branched C₁-C₆ haloalkoxy;
R₂ and R₃ are independently selected from the group consisting of hydrogen, halogen, amine, hydroxy, and alkyl C₁-C₆ linear or branched, alkoxy C₁-C₆ linear or branched;
R₄ is:

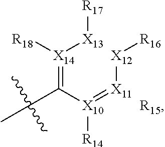

wherein:
X₁₀, X₁₁, X₁₂, X₁₃, and X₁₄ are independently selected from the group consisting of carbon, CH, or nitrogen; where:
when at least one of the substituents X₁₀-X₁₄ is N or CH, the corresponding R (R₁₄—R₁₈) is null,
R₁₄, R₁₅, R₁₆, R₁₇, and R₁₈ are independently selected from the group consisting of hydrogen, halogen, hydroxy(alkyl C₂-C₄ linear or branched), alkyl C₁-C₆ linear or branched, alkoxy C₁-C₆ linear or branched;
R₄ and R₈ are optionally substituted to form a piperidine, homopiperidine, homomorpholine, or pyrrolidine;
R₅ and R₆ are independently selected from the group consisting of hydrogen, linear or branched C₁-C₆ alkyl; and
R₇ and R₈ are independently selected from the group consisting of hydrogen, linear or branched C₁-C₆ alkyl.

Additionally, the present invention further refers to compositions comprising one or more compound(s) of Formula (I) or a salt, hydrate, solvate and pharmaceutically acceptable isomer thereof; and one or more pharmaceutically acceptable excipients.

In addition, kits in accordance with this invention may comprise such compositions and application devices, which may include ampoules, syringes and others. Alternatively, the kits according to this invention comprise more than one compound of Formula (I) arranged in one or more dosage forms, including without limitation, tablets, accompanied by administration instructions.

The present invention further refers to methods of treatment, prevention, relief, suppression and/or control of diseases related to neuropathic pain. Uses of Formula (I) compound(s) to prepare a drug for the treatment of pathologies related to neuropathic pain are also taught. Finally, the present invention teaches processes for obtaining compound(s) of Formula (I).

### DETAILED DESCRIPTION OF THE INVENTION

The present invention presents, in a first embodiment, the compound of Formula (I):

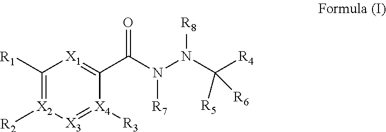

or a pharmaceutically acceptable salt, hydrate, solvate, ester and isomer thereof, wherein:
   X₁, X₂, X₃, and X₄ are independently selected from the group consisting of carbon, CH, or nitrogen, wherein:
   when at least one of the substituents X₂, X₄ is N or CH, the corresponding R (R₂, R₃) is null;
   R₁ is:

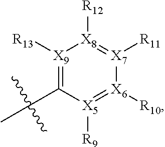

where:
X₅, X₆, X₇, X₈, and X₉ are independently selected from the group consisting of carbon, CH, or nitrogen, where:
when at least one of the substituents X₅-X₉ is N or CH, the corresponding R (R₉₋₁₃) is null;
R₉, R₁₀, R₁₁, R₁₂, and R₁₃ are independently selected from the group consisting of hydrogen, halogen, linear or branched C₁-C₆ alkyl, linear or branched C₁-C₆ alkoxy, C₃-C₇ cycloalkoxy, difluoromethyl, trifluoromethyl, linear or branched C₁-C₆ haloalkoxy;
R₂ and R₃ are independently selected from the group consisting of hydrogen, halogen, amine, hydroxy, and alkyl C₁-C₆ linear or branched, alkoxy C₁-C₆ linear or branched;
R₄ is:

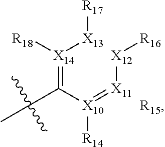

wherein:
X₁₀, X₁₁, X₁₂, X₁₃, and X₁₄ are independently selected from the group consisting of carbon, CH, or nitrogen, wherein:
when at least one of the substituents X₁₀-X₁₄ is N or CH, the corresponding R (R₁₄—R₁₈) is null,
R₁₄, R₁₅, R₁₆, R₁₇, and R₁₈ are independently selected from the group consisting of hydrogen, halogen, hydroxy(alkyl C₂-C₄ linear or branched), alkyl C₁-C₆ linear or branched, alkoxy C₁-C₆ linear or branched;
R₄ and R₈ are optionally substituted to form together a piperidine, homopiperidine, homomorpholine, or pyrrolidine, whereby R₁₈ is null;
R₅ and R₆ are independently selected from the group consisting of hydrogen, linear or branched C₁-C₆ alkyl; and
R₇ and R₈ are independently selected from the group consisting of hydrogen, linear or branched C₁-C₆ alkyl.

As described herein, the compounds of the present invention comprise multiple variable groups (R, X₁, etc.). As a person skilled in the art will recognize, the group combinations contemplated by this invention are those combinations that result in the formation of stable or chemically viable compounds. The term “stable” in this context refers to compounds that are not substantially altered when subjected to conditions that allow their production, detection and preferably their recovery, purification and use for one or more of the purposes disclosed herein. In some embodiments, a stable compound or chemically viable compound is one that is not substantially altered when kept at a temperature of 40° C. or less, in the absence of moisture or other chemically reactive conditions, for at least one week.

Additionally, the chemical structures drawn herein are intended to be understood as they would be understood by a person skilled in the art. For example, with respect to the formulas III, IV, X, a person skilled in the art would understand that X₂ and X₃ are connected by a simple bond, even though the bonds between these groups can be hidden by the labels of the atoms in the chemical structures.

In an embodiment, X₁, X₂, X₃, and X₄ are independently selected from the group consisting of carbon, CH, or nitrogen, where when at least one of the substituents X₂, X₄ is N or CH, the corresponding R (R₂, R₃) is null; R₁ is:

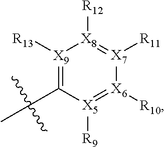

R₉, where:
X₅, X₆, X₇, X₈, and X₉ are independently selected from the group consisting of carbon, CH, or nitrogen, where when at least one of the substituents X₅-X₉ is N or CH, the corresponding R (R₉-. R₁₃) is null; R₉, R₁₀, R₁₁, R₁₂, and R₁₃ are independently selected from the group consisting of hydrogen, halogen, C₁-C₆ linear or branched alkyl, C₁-C₆ linear or branched alkoxy, C₃-C₇ cycloalkoxy, difluoromethyl, trifluoromethyl, haloalkoxy C₁-C₆ linear or branched; R₂ and R₃ are independently selected from the group consisting of hydrogen, halogen, amine, hydroxy, and alkyl C₁-C₆ linear or branched, alkoxy C₁-C₆ linear or branched; R₄ is:

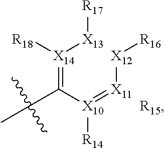

where:
X₁₀, X₁₁, X₁₂, X₁₃ and X₁₄ are independently selected from the group consisting of carbon, CH, or nitrogen, where when at least one of the substituents X₁₀-X₁₄ is N or CH, the corresponding R (R₁₄—R₁₈) is null; R₁₄, R₁₅, R₁₆, R₁₇, and R₁₈ are independently selected from the group consisting of hydrogen, halogen, hydroxy(alkyl C₂₋₄ linear or branched), C₁-C₆ linear or branched alkyl, C₁-C₆ linear or branched alkoxy; R₄ and R₈ are optionally substituted to form a piperidine, homopiperidine, homomorpholine, or a pyrrolidine; R₅ and R₆ are independently selected from the group consisting of hydrogen, linear or branched C₁-C₆ alkyl, and R₇ and R₈ are independently selected from the group consisting of hydrogen, linear or branched C₁-C₆ alkyl.

In an embodiment, X₁, X₂, X₃, and X₄ are independently selected from the group consisting of carbon and nitrogen.

In an embodiment, R₁ is:

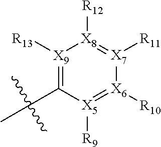

wherein X₅, X₆, X₇, X₈, and X₉ are independently selected from the group consisting of carbon and nitrogen. Preferably, X₅, X₆, X₇, and X₉ are carbon and X₈ is selected from carbon and nitrogen.

In an embodiment, R₉, R₁₀, R₁₁, R₁₂, and R₁₃ are independently selected from the group consisting of hydrogen, fluorine, chlorine, bromine, methyl, ethyl, propyl, isopropyl, butyl, sec-butyl, tert-butyl, methoxyl, ethoxyl, propoxyl, isopropoxyl, butoxyl, sec-butoxyl, tert-butoxyl, cyclopropoxyl, cyclobutoxyl, cyclopentoxyl, difluoromethyl, trifluoromethyl, difluoromethoxyl and trifluoromethoxyl. Preferably, R₉ is selected from the group consisting of hydrogen, fluorine, and methyl; R₁₀ is hydrogen; R₁₁ is selected from the group consisting of chlorine, methoxyl, ethoxyl, isopropoxyl, cyclopropoxyl, and difluoromethoxyl; R₁₂ is hydrogen or absent when X₈ is nitrogen; and R₁₃ is selected from the group consisting of hydrogen and methyl.

In an embodiment, R₂ and R₃ are independently selected from the group consisting of hydrogen, fluorine, chlorine, bromine, amine, hydroxy, methyl, ethyl, propyl, isopropyl, butyl, sec-butyl, tert-butyl, methoxyl, ethoxyl, propoxyl, isopropoxyl, butoxyl, sec-butoxyl and tert-butoxyl; or absent when the corresponding substituent X₂ and X₄ is nitrogen. Preferably, R₂ is hydrogen or absent when X₂ is nitrogen; and R₃ is hydrogen or absent when X₄ is nitrogen.

In an embodiment, R₄ is:

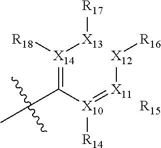

wherein X₁₀, X₁₁, X₁₂, X₁₃, and X₁₄ are independently selected from the group consisting of carbon and nitrogen. Preferably, X₁₀, X₁₁, X₁₂ and X₁₄ are carbon and X₁₃, is selected from carbon and nitrogen.

In an embodiment, R₁₄, R₁₅, R₁₆, R₁₇, and R₁₈ are independently selected from the group consisting of hydrogen, fluorine, chlorine, bromine, 1-hydroxyethyl, 2-hydroxyethyl, methyl, ethyl, propyl, isopropyl, butyl, sec-butyl, tert-butyl, methoxyl, ethoxyl, propoxyl, isopropoxyl, butoxyl, sec-butoxyl and tert-butoxyl; or null when R₄ and R₈ together form a piperidine homopiperidine, homomorpholine, or pyrrolidine. Preferably, R₁₄ is selected from the group consisting of hydrogen and fluorine; R₁₅ is selected from the group consisting of hydrogen, methoxyl, ethoxyl, and 2-hydroxyethyl; R₁₆ is selected from the group consisting of hydrogen, chlorine, methoxyl; R₁₇ is selected from the group consisting of hydrogen, methoxyl, ethoxyl, or absent when X₁₃ is nitrogen; and R₁₈ is selected from the group consisting of hydrogen, fluorine, methyl, or null when R₄ and R₈ together form a piperidine homopiperidine, homomorpholine, or pyrrolidine.

In one embodiment, R₄ and R₈ are optionally substituted to form together a piperidine, homopiperidine, homomorpholine, or pyrrolidine, where R₁₈ is null.

In one embodiment, R₅ and R₆ are independently selected from the group consisting of hydrogen, methyl, ethyl, propyl, isopropyl, butyl, sec-butyl, and tert-butyl. Preferably, R₅ and R₆ are independently selected from the group consisting of hydrogen and methyl.

In one embodiment, R₇ and R₈ are independently selected from the group consisting of hydrogen, methyl, ethyl, propyl, isopropyl, butyl, sec-butyl, and tert-butyl. Preferably, R₇ and R₈ are independently selected from the group consisting of hydrogen and methyl.

In an implementation of the first embodiment, the Formula (I) compound(s) is(are) selected from the group(s) consisting of:
N′-(3,5-dimethoxybenzyl)-6-(4-methoxyphenyl)pyrazine-2-carbohydrazide (Compound 1);
N′-(4-chlorobenzyl)-6-(4-methoxyphenyl)pyrazine-2-carbohydrazide (Compound 2);
N′-(3,5-dimethoxybenzyl)-6-(4-isopropoxyphenyl)pyrazine-2-carbohydrazide (Compound 3);
N′-(4-chlorobenzyl)-6-(4-cyclopropoxyphenyl)pyrazine-2-carbohydrazide (Compound 4);
N′-(4-chlorobenzyl)-6-(4-chlorophenyl)pyrazine-2-carbohydrazide (Compound 5);
N-(6-chloro-3,4-dihydroisoquinoline-2 (1H)-yl)-6-(4-chlorophenyl)pyrazine-2-carboxamide (Compound 6);
6-(4-(difluoromethoxy)phenyl)-N′-(3,5-dimethoxybenzyl)pyrazine-2-carbohydrazide (Compound 7);
6-(6-(difluoromethoxy)pyridin-3-yl)-N′-(3,5-dimethoxybenzyl)pyrazine-2-carbohydrazide (Compound 8);
N′-(4-chlorobenzyl)-6-(4-ethoxyphenyl)pyrazine-2-carbohydrazide (Compound 9);
N′-(3,5-dimethoxybenzyl)-6-(4-ethoxyphenyl)pyrazine-2-carbohydrazide (Compound 10);
6-(4-ethoxyphenyl)-N-(8-methoxy-1,3,4,5-tetrahydro-2H-benzo[c]azepin-2-yl)pyrazine-2-carboxamide (Compound 11);
6-(4-ethoxyphenyl)-N-(7-methoxy-2,3-dihydrobenzo[f][1,4]oxazepine-4(5H)-yl)pyrazine-2-carboxamide (Compound 12);
6-(4-ethoxyphenyl)-N-(5-methoxyisoindolin-2-yl)pyrazine-2-carboxamide (Compound 13);
6-(4-ethoxyphenyl)-N′-(5-methoxy-2-methylbenzyl)pyrazine-2-carbohydrazide (Compound 14);
6-(4-ethoxyphenyl)-N′-(2-fluoro-5-methoxybenzyl)pyrazine-2-carbohydrazide (Compound 15);
6-(4-ethoxyphenyl)-N′-(3-methoxybenzyl)pyrazine-2-carbohydrazide (Compound 16);
N′-(3,5-dimethoxybenzyl)-6-(4-ethoxyphenyl)-N′-methylpyrazine-2-carbohydrazide (Compound 17);
6-(4-ethoxyphenyl)-N′-((5-methoxy-2-methylpyridin-3-yl)methyl)pyrazine-2-carbohydrazide (Compound 18);
6-(4-ethoxyphenyl)-N′-(3-(1-hydroxyethyl)-5-methoxybenzyl)pyrazine-2-carbohydrazide (Compound 19);
6-(4-ethoxyphenyl)-N′-((2-fluoro-5-(1-hydroxyethyl)pyridin-3-yl)methyl)pyrazine-2-carbohydrazide (Compound 20);
6-(4-ethoxyphenyl)-N′-(2-fluoro-5-(1-hydroxyethyl)benzyl)pyrazine-2-carbohydrazide (Compound 21);
6-(4-ethoxyphenyl)-N′-(2-fluorobenzyl)pyrazine-2-carbohydrazide (Compound 22);
6-(4-ethoxyphenyl)-N′-(3-(1-hydroxyethyl)benzyl)pyrazine-2-carbohydrazide (Compound 23);
(S)—N′-(1-(3,5-dimethoxyphenyl)ethyl)-6-(4-ethoxyphenyl)pyrazine-2-carbohydrazide (Compound 24);
(R)—N′-(1-(3,5-dimethoxyphenyl)ethyl)-6-(4-ethoxyphenyl)pyrazine-2-carbohydrazide (Compound 25);
6-(4-ethoxyphenyl)-N′-((2-fluoro-5-methoxypyridin-3-yl)methyl)pyrazine-2-carbohydrazide (Compound 26);
N′-(3,5-dimethoxybenzyl)-6-(4-ethoxy-2-fluorophenyl)pyrazine-2-carbohydrazide (Compound 27);
N′-(3,5-dimethoxybenzyl)-6-(4-ethoxy-2-methylphenyl)pyrazine-2-carbohydrazide (Compound 28);
N′-(3,5-dimethoxybenzyl)-6-(6-ethoxypyridin-3-yl)pyrazine-2-carbohydrazide (Compound 29);
6-(6-ethoxypyridin-3-yl)-N′-(3-methoxybenzyl)pyrazine-2-carbohydrazide (Compound 30);
6-(6-ethoxypyridin-3-yl)-N′-((2-fluoro-5-methoxypyridin-3-yl)methyl)pyrazine-2-carbohydrazide (Compound 31);
6-(6-ethoxypyridin-3-yl)-N′-(2-fluoro-5-methoxybenzyl)pyrazine-2-carbohydrazide (Compound 32);
6-(6-ethoxypyridin-3-yl)-N′-(5-methoxy-2-methylbenzyl)pyrazine-2-carbohydrazide (Compound 33);
6-(6-ethoxypyridin-3-yl)-N′-((5-methoxy-2-methylpyridin-3-yl)methyl)pyrazine-2-carbohydrazide (Compound 34);
6-(6-ethoxypyridin-3-yl)-N′-(3-(1-hydroxyethyl)benzyl)pyrazine-2-carbohydrazide (Compound 35);
6-(6-ethoxypyridin-3-yl)-N′-(3-(1-hydroxyethyl)-5-methoxybenzyl)pyrazine-2-carbohydrazide (Compound 36);
6-(6-ethoxypyridin-3-yl)-N′-((2-fluoro-5-(1-hydroxyethyl)pyridin-3-yl)methyl)pyrazine-2-carbohydrazide (Compound 37);
6-(6-ethoxypyridin-3-yl)-N′-(2-fluoro-5-(1-hydroxyethyl)benzyl)pyrazine-2-carbohydrazide (Compound 38);
6-(6-ethoxypyridin-3-yl)-N′-(2-fluorobenzyl)pyrazine-2-carbohydrazide (Compound 39);
N′-(3,5-dimethoxybenzyl)-6-(6-ethoxy-4-methylpyridin-3-yl)pyrazine-2-carbohydrazide (Compound 40);
N′-(3,5-dimethoxybenzyl)-6-(6-ethoxy-2-methylpyridin-3-yl)pyrazine-2-carbohydrazide (Compound 41);
6-(6-cyclopropoxypyridin-3-yl)-N′-(3,5-dimethoxybenzyl)pyrazine-2-carbohydrazide (Compound 42);
N′-(3,5-dimethoxybenzyl)-6-(6-isopropoxypyridin-3-yl)pyrazine-2-carbohydrazide (Compound 43);
4-(4-chlorophenyl)-N′-(3,5-dimethoxybenzyl)pyrimidine-2-carbohydrazide (Compound 44);
6-(4-ethoxyphenyl)-N-(7-methoxy-3,4-dihydroisoquinoline-2(1H)-yl)pyrazine-2-carboxamide (Compound 45);
6-(4-ethoxyphenyl)-N′-(2-fluorobenzyl)pyridazine-4-carbohydrazide (Compound 46);
5-(4-ethoxyphenyl)-N′-(2-fluorobenzyl)pyridazine-3-carbohydrazide (Compound 47);
4-(4-ethoxyphenyl)-N′-(2-fluorobenzyl)picolinohydrazide (Compound 48);
5-(4-ethoxyphenyl)-N′-(2-fluorobenzyl)nicotinohydrazide (Compound 49);
6-(4-ethoxyphenyl)-N′-(2-fluorobenzyl)picolinohydrazide (Compound 50);
2-(4-ethoxyphenyl)-N′-(2-fluorobenzyl)isonicotinohydrazide (Compound 51);
2-(4-ethoxyphenyl)-N′-(2-fluorobenzyl)pyrimidine-4-carbohydrazide (Compound 52) and
4-(4-ethoxyphenyl)-N′-(2-fluorobenzyl)pyrimidine-2-carbohydrazide (Compound 53).

In an implementation of the first embodiment, the Formula (I) compound(s) are voltage-gated sodium channel blockers Nav 1.7 and/or Nav 1.8. In a preferred embodiment, the Formula (I) compounds are dual voltage-gated sodium channel blockers Nav 1.7 and Nav 1.8.

In an implementation of the first embodiment, the compound(s) of Formula (I) may have a basic nature and, consequently, pharmaceutically acceptable salts may be obtained by the addition of organic or inorganic acids. Non-limiting examples of organic acids that can be used are fumaric, maleic, benzoic, lactic acids, among others. Among the inorganic acids, we can mention hydrochloric acid, phosphoric acid, hydrobromic acid, sulfuric acid, nitric acid, among others.

In an implementation of the first embodiment, the compound(s) of Formula (I) can be obtained in the form of crystals, which can be optionally presented as pharmaceutically acceptable solvates, in which the solvent is incorporated in stoichiometric proportions or not into the crystal lattice. In further embodiments, the crystallization solvent is water, resulting in pharmaceutically acceptable hydrates.

Finally, in an implementation of the first embodiment, the Formula (I) compound(s) may present more than one isomer, including without limitation, spatial isomerism, such as geometric and optical isomerism.

### Definitions

In order to clarify or elucidate the terms used in this invention, the following definitions are presented, whereby the scope is not limited thereto.

The term “halogen” refers to the elements of the 7A family of the periodic table, which are: fluorine (F), chlorine (Cl), bromine (Br), iodine (I), astatine (At) and tennessine (Ts).

The term “linear or branched C₁-C₆ alkyl refers to the saturated hydrocarbon groups of linear or branched chain, such as methyl, ethyl, propyl, isopropyl, isobutyl, butyl, sec-butyl, and tert-butyl but not being limited to the same.

The term “linear or branched C₁-C₆ alkoxy” refers to alkyl groups attached to a radical oxygen, such as methoxyl, ethoxyl, propoxyl, isopropoxyl, butoxyl, sec-butoxyl, and tert-butoxyl but not being limited to the same.

The term “cycloalkoxy C₃-C₇” refers to alkyl cycle groups attached to a radical oxygen such as cyclopropoxyl, cyclobutoxyl, cyclopentoxyl and cyclohexyl, but not being limited to the same.

The term “linear or branched haloalkoxy C₁-C₆” refers to alkyl groups attached to a radical oxygen and at least one halogen, such as, but not limited to, chloromethoxyl, dichloromethoxyl, trichloromethoxyl, fluoromethoxyl, difluoromethoxyl and trifluoromethoxyl.

The term “hydroxy(alkyl C₂-C₄ linear or branched)” refers to saturated, linear or branched alkyl groups replaced with at least one hydroxyl, such as 1-hydroxyethyl, 2-hydroxyethyl, 1-hydroxypropyl, 2-hydroxypropyl, 3-hydroxypropyl, hydroxyisopropyl, hydroxyisobutyl, 1-hydroxybutyl, 2-hydroxybutyl, 3-hydroxybutyl, 4-hydroxybutyl, hydroxy-sec-butyl and hydroxy-tert-butyl, but not being limited to the same.

In a second embodiment, the present invention presents a composition comprising a therapeutically effective amount of compound(s) of Formula (I) of the present invention or a pharmaceutically acceptable salt, hydrate, solvate, and isomer thereof; and one or more pharmaceutically acceptable excipients.

Pharmaceutically acceptable excipients are any substance, other than the active pharmaceutical ingredient, that has been evaluated for its safety and that is intentionally added to the dosage form. Such excipients are selected according to the pharmaceutical dosage form of interest, its route of administration, physicochemical compatibility with the active ingredient, and the effect on efficacy.

In addition, these excipients are widely known in the state of the art and are classified according to their function, including without limitation diluents, binders, disintegrants or disaggregators, lubricants, suspending agents, thickeners, solvents, surfactants, sliders, anti-caking agents or flow agents, glazing agents, plasticizers, sweeteners, isotonicity agents, dyes and pigments, preservatives, antioxidants, modifying agents or pH control, complexing agents, chelating agents, flavorings, viscosity modifying agents, opacifiers, permeation promoters, among others.

In an implementation of the second embodiment, pharmaceutical compositions can be administered by several routes including oral, sublingual, nasal, parenteral, injectable, submuscular, topical, transdermal, ocular, rectal, but not limited to the same.

In a third embodiment, the present invention presents the use of compound(s) of Formula (I) or a salt, hydrate, solvate and pharmaceutically acceptable isomer thereof to prepare a drug to treat pathologies related to neuropathic pain.

In an implementation of the third embodiment, said pathologies are selected from the group consisting of peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia and postsurgical neuralgia.

In a fourth embodiment, the present invention presents a method of treatment, prevention, relief, suppression and/or control of pathologies related to neuropathic pain comprising the administration of an effective amount of Formula (I) compound(s) or a salt, hydrate, solvate and pharmaceutically acceptable isomer thereof.

In an implementation of the fourth embodiment, the method is for the treatment, prevention, relief, suppression and/or control of peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia and postsurgical neuralgia.

In another implementation of the fourth embodiment, the administration of at least one Formula (I) compound is selected from the group comprising oral, sublingual, nasal, parenteral, injectable, submuscular, topical, transdermal, ocular and rectal routes.

In a fifth embodiment of the invention, processes for obtaining Formula (I) compounds are presented, comprising the following steps:
(a) formation of the Formula IV or Formula VI intermediate:

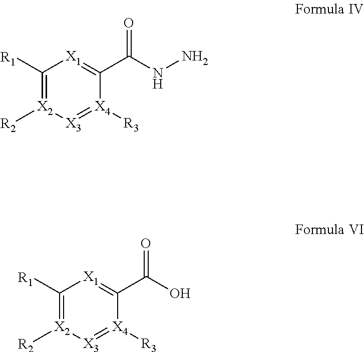

from the hydrazinolysis reaction or hydrolysis of a Formula V intermediate:

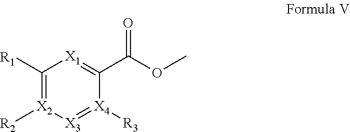

(b) formation of the Formula II intermediate:

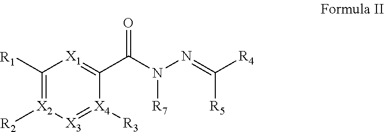

from condensation between Formula III intermediates

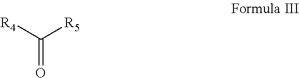

and Formula IV intermediates with or without the presence of a catalyst and a suitable solvent;
(c) Formula I compound is obtained:

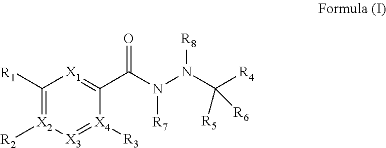

from the reduction of Formula II intermediates or from reactions and amide coupling between intermediates VI and VII with or without the presence of a catalyst and a suitable solvent:

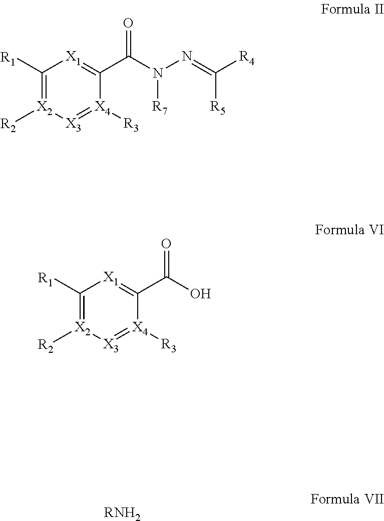

wherein:
   X₁, X₂, X₃, and X₄ are independently selected from the group consisting of carbon, CH, or nitrogen, wherein:
   when at least one of the substituents X₂, X₄ is N or CH, the corresponding R (R₂, R₃) is null;
   R₁ is:

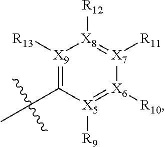

wherein:
X₅, X₆, X₇, X₈, and X₉ are independently selected from the group consisting of carbon, CH, or nitrogen, wherein:
when at least one of the substituents X₅-X₉ is N or CH, the corresponding R (R₉—R₁₃) is null;
R₉, R₁₀, R₁₁, R₁₂, and R₁₃ are independently selected from the group consisting of hydrogen, halogen, linear or branched C₁-C₆ alkyl, linear or branched C₁-C₆ alkoxy, cycloalkoxy C₃-C₇, difluoromethyl, trifluoromethyl, linear or branched C₁-C₆ haloalkoxy;
R₂ and R₃ are independently selected from the group consisting of hydrogen, halogen, amine, hydroxy, and alkyl C₁-C₆ linear or branched, alkoxy C₁-C₆ linear or branched;
R₄ is:

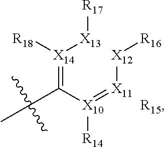

wherein:
X₁₀, X₁₁, X₁₂, X₁₃, and X₁₄ are independently selected from the group consisting of carbon, CH, or nitrogen, wherein:
when at least one of the substituents X₁₀-X₁₄ is N or CH, the corresponding R (R₁₄—R₁₈) is null,
R₁₄, R₁₅, R₁₆, R₁₇, and R₁₈ are independently selected from the group consisting of hydrogen, halogen, hydroxy(alkyl C₂-C₄ linear or branched), alkyl C₁-C₆ linear or branched, alkoxy C₁-C₆ linear or branched;
R₄ and R₈ are optionally substituted to form a piperidine, homopiperidine, homomorpholine, or pyrrolidine;
R₅ and R₆ are independently selected from the group consisting of hydrogen, linear or branched C₁-C₆ alkyl; and
R₇ and R₈ are independently selected from the group consisting of hydrogen, linear or branched C₁-C₆ alkyl.

In an implementation of the fifth embodiment, the process additionally comprises a step (d) of formation of compound(s) of Formula (I) in which R₇ is methyl from the reaction of N-alkylation of a compound obtained in step (c) with formaldehyde.

In an implementation of the fifth embodiment, step (b) the compound of Formula III is a derivative of acetophenone. In another embodiment, the compound of Formula III is a derivative of benzaldehyde.

In an implementation of the fifth embodiment, the reducing agent of step (c) is selected between triethylsilane or sodium borohydride. In another embodiment, in step (c) the acid is selected as TFA.

The compound(s) of Formula (I) of this invention have been prepared from the synthetic route described in General Scheme 1. However, those skilled in the art will readily notice that additional detailing and/or modifications to the arrangement of one or more steps can be accomplished without departing from the processes taught here. Such variations can be, without limitation, combinations of solvents and catalysts, including stereoselective ones, protective groups, among others.

In the following General Scheme 1, the formation of the Formula II, III, IV and V intermediates is described in addition to the formation of the Formula (I) compound(s), R₇ can be hydrogen or an alkyl group C₁₋₆.

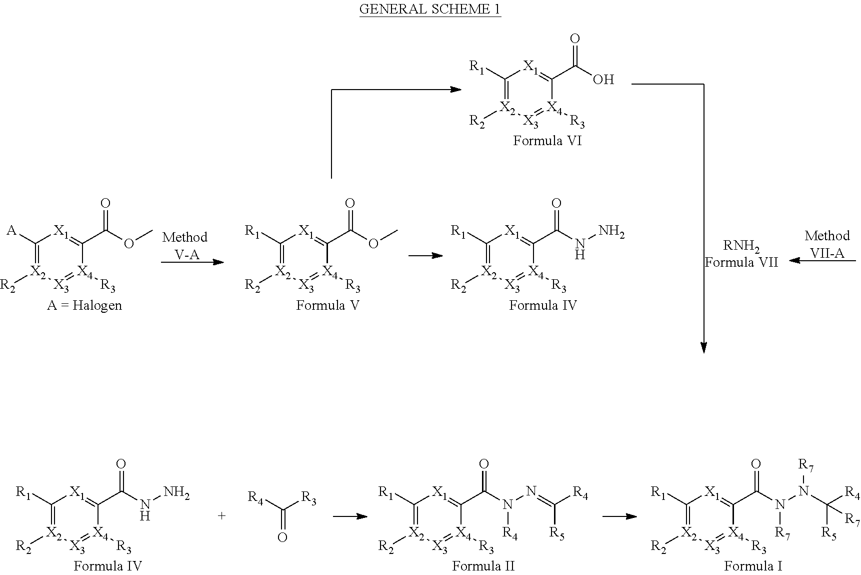

As illustrated in General Scheme 1, Formula I compounds can be prepared from reduction reactions of Formula II intermediates without or by means of acid catalysis using concentrated hydrochloric acid, acetic acid, trifluoroacetic acid, or formic acid in suitable solvents such as THF. Formula II intermediates can be prepared by condensation reaction between Formula IV hydrazides and Formula III aldehydes or ketones. Formula IV intermediates can be prepared through hydrazinolysis reactions from the corresponding esters (Formula V) obtained following the methods described below. Formula III intermediates can be prepared from the corresponding reagents following the methods described below, some of which can be obtained commercially.

### Formula V Intermediates:

Following General Scheme 1, the intermediates of Formula V are presented, however, such methods are not limiting.

### Method V-A

### Intermediates V-1 to IV-18:

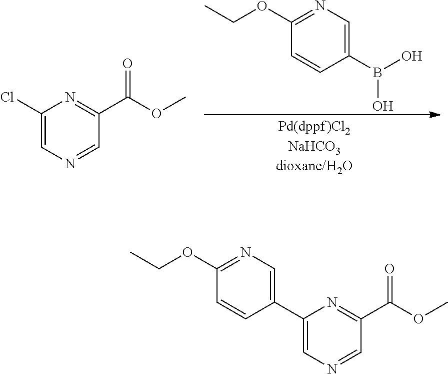 [SMILES: COC(=O)c1cncc(Cl)n1 ; CCOc1ccc(B(O)O)cn1 ; CCOc1ccc(-c2cncc(C(=O)OC)n2)cn1]

In a round-bottomed flask containing methyl 6-chloropyrazine-2-carboxylate (26.0 mmol) in dioxane (50.0 mL) and H₂O (10.0 mL), boronic acid 6-(ethoxy-pyridin-3-yl) (28.6 mmol), NaHCO₃ (39.1 mmol) and Pd(dppf)Cl₂ (1.30 mmol) were added under an atmosphere of N₂. The reaction mixture was kept under agitation at 70° C. for 12 hours. After full consumption of the starting reagent (monitored by CCD), the mixture was concentrated under reduced pressure to remove the solvent. The residue obtained was diluted with H₂O (100 mL) and extracted with AcOEt (100 mL×3). The organic phase was concentrated under reduced pressure and purified by chromatography column (petroleum ether/AcOEt: 50/1→0/1). 4.50 g (61.3% yield) of the corresponding ester were obtained in the form of a white solid.

TABLE 1
Intermediates obtained from the corresponding reagents
following Method V-A.

| Intermediate | Structure |
|---|---|
| V-1 | 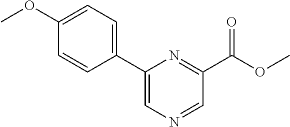 |
| V-2 | 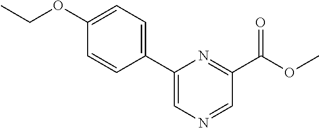 [SMILES: CCOc1ccc(-c2cncc(C(=O)OC)n2)cc1] |
| V-3 | 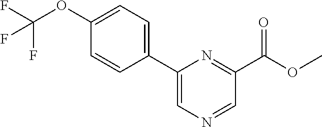 [SMILES: COC(=O)c1cncc(-c2ccc(OC(F)(F)F)cc2)n1] |
| V-4 | 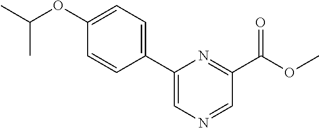 [SMILES: COC(=O)c1cncc(-c2ccc(OC(C)C)cc2)n1] |
| V-5 | 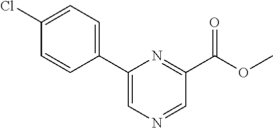 [SMILES: COC(=O)c1cncc(-c2ccc(Cl)cc2)n1] |
| V-6 | 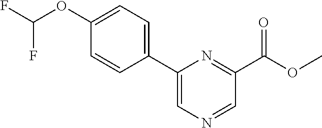 [SMILES: COC(=O)c1cncc(-c2ccc(OC(F)F)cc2)n1] |
| V-7 | 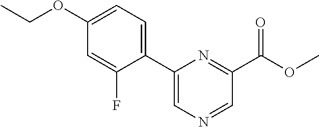 [SMILES: CCOc1ccc(-c2cncc(C(=O)OC)n2)c(F)c1] |
| V-8 | 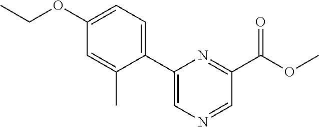 [SMILES: CCOc1ccc(-c2cncc(C(=O)OC)n2)c(C)c1] |
| V-9 | 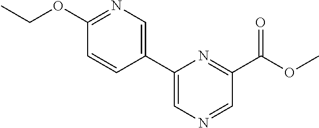 [SMILES: CCOc1ccc(-c2cncc(C(=O)OC)n2)cn1] |
| V-10 | 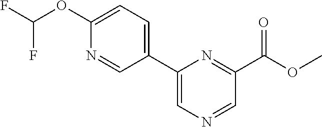 [SMILES: COC(=O)c1cncc(-c2ccc(OC(F)F)nc2)n1] |
| V-11 | 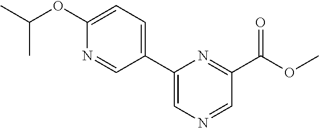 [SMILES: COC(=O)c1cncc(-c2ccc(OC(C)C)nc2)n1] |
| V-12 | 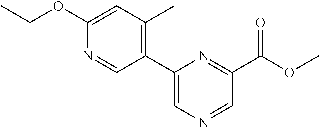 [SMILES: CCOc1cc(C)c(-c2cncc(C(=O)OC)n2)cn1] |
| V-13 | 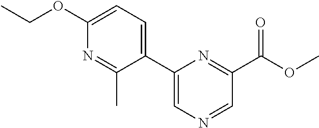 [SMILES: CCOc1ccc(-c2cncc(C(=O)OC)n2)c(C)n1] |
| V-14 | 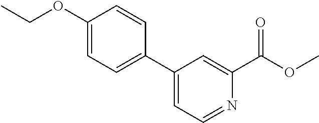 [SMILES: CCOc1ccc(-c2ccnc(C(=O)OC)c2)cc1] |
| V-15 | 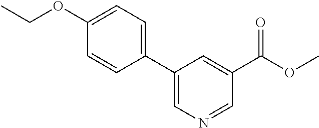 [SMILES: CCOc1ccc(-c2cncc(C(=O)OC)c2)cc1] |
| V-16 | 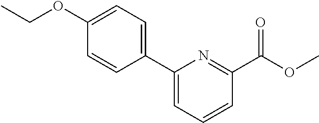 [SMILES: CCOc1ccc(-c2cccc(C(=O)OC)n2)cc1] |
| V-17 | 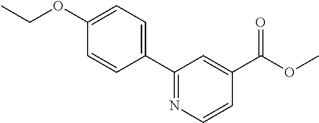 [SMILES: CCOc1ccc(-c2cc(C(=O)OC)ccn2)cc1] |
| V-18 | 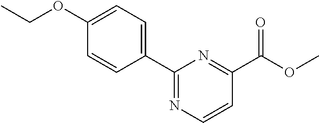 [SMILES: CCOc1ccc(-c2nccc(C(=O)OC)n2)cc1] |

### Method V-B

### Intermediate V-19

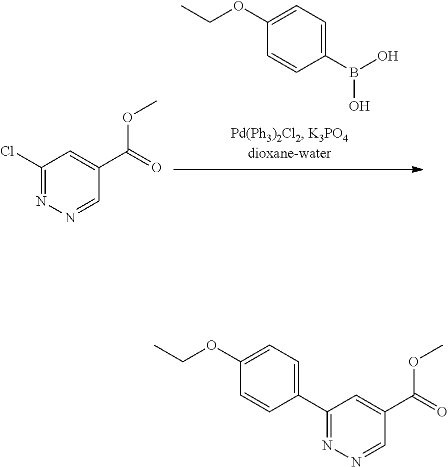 [SMILES: COC(=O)c1cnnc(Cl)c1 ; CCOc1ccc(B(O)O)cc1 ; CCOc1ccc(-c2cc(C(=O)OC)cnn2)cc1]

In a round-bottomed flask containing methyl 6-chloropyridazine-4-carboxylate (17.4 mmol) and (4-ethoxyphenyl)boronic acid (20.8 mmol) in dioxane (10.0 mL) and water (10 mL), K₃PO₄ (43.5 mmol) was added. The reaction mixture was degassed with N₂ and then bis(triphenylphosphine)palladium chloride (8.7 mmol) was added at room temperature. The reaction was maintained under agitation at 60° C. for 16 hours. After total consumption of the starting reagent (monitored by CCD), the mixture was filtered in celite and concentrated. The residue was diluted in water and extracted with AcOEt. The organic phase was concentrated and purified by chromatography column (petroleum ether/AcOEt 85/15) providing the product of interest in the form of a greenish solid at 40% yield (1.8 g).

### V-C Method

### Step V-C-I:

### Precursor V-20I:

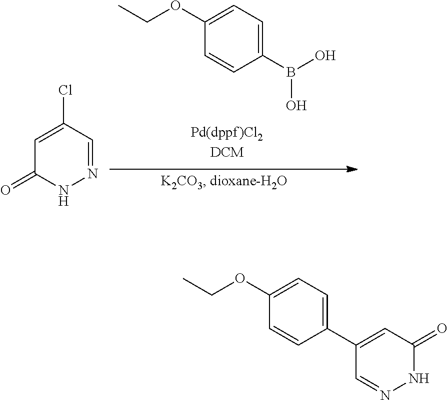 [SMILES: O=c1cc(Cl)cn[nH]1 ; CCOc1ccc(B(O)O)cc1 ; CCOc1ccc(-c2cn[nH]c(=O)c2)cc1]

In a round-bottomed flask containing 5-chloropyridazine-3(2H)-one (45.9 mmol) in dioxane (120.0 mL) and water (12 mL), (4-ethoxyphenyl) boronic acid (45.9 mmol), K₂CO₃ (68.9 mmol) and Pd(dppf)Cl₂·DCM (4.59 mmol) was added. The reaction was maintained under agitation at 90° C. for 16 hours. After total consumption of the starting reagent (monitored by CCD), the mixture was cooled to room temperature, diluted in ice water (50 mL) and extracted with AcOEt (150 mL×2). The organic phase was concentrated resulting in a yellow solid. The compound obtained was used in the next step without further purification.

### Step V-C-II:

### Precursor V-20II:

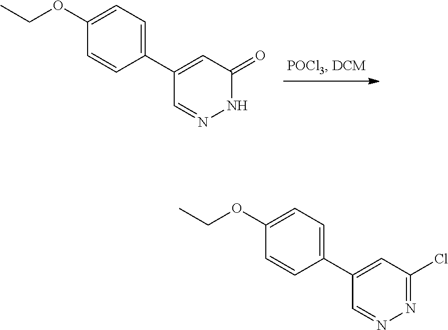 [SMILES: CCOc1ccc(-c2cn[nH]c(=O)c2)cc1 ; CCOc1ccc(-c2cnnc(Cl)c2)cc1]

In a round-bottomed flask containing 5-(4-ethoxyphenyl) pyridazin-3 (2H)-one (23.1 mmol) in DCM (100.0 mL), phosphoryl chloride (34.7 mmol) was added at 0° C. The mixture was heated to 80° C. and kept under agitation for 16 hours. After total consumption of the starting reagent (monitored by CCD), the mixture was concentrated under reduced pressure, diluted in ice water (50 mL) and extracted with AcOEt (200 mL×2). The organic phase was concentrated resulting in a yellow solid. The compound obtained was used in the next step without further purification.

### Step V-C-III:

### Intermediate V-20:

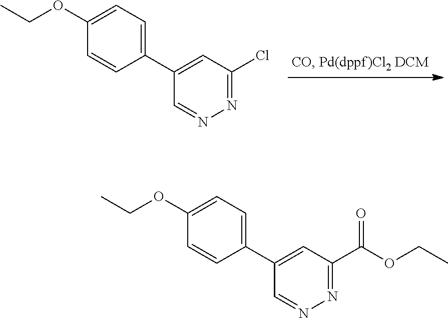 [SMILES: CCOc1ccc(-c2cnnc(Cl)c2)cc1 ; CCOC(=O)c1cc(-c2ccc(OCC)cc2)cnn1]

In a round-bottomed flask containing 3-chloro-5-(4-ethoxyphenyl) pyridazine (8.5 mmol) in ethanol (50.0 mL), triethylamine (3.59 mmol) and Pd(dppf)Cl₂·DCM (0.85 mmol) were added at room temperature and CO (100 Psi) was applied in an autoclave. The mixture was heated to 90° C. and kept under agitation for 16 hours. After total consumption of the starting reagent (monitored by CCD), the mixture was concentrated under reduced pressure, diluted in ice water (50 mL) and extracted with AcOEt (100 mL×2). The organic phase was concentrated resulting in a brown solid. 1.6 g of the product of interest were obtained with a yield of 68.9%.

### Method V-D

### Step V-D-I:

### Precursors V-21I and V-22I:

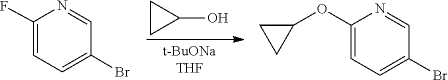

In a round-bottomed flask containing 5-bromo-2-fluoropyridine (28.4 mmol) in THF (50.0 mL), cyclopropanol (42.62 mmol) and t-BuONa (42.6 mmol) were added. The mixture was heated to 90° C. and kept under agitation for 16 hours. After total consumption of the starting reagent (monitored by LC-MS), the mixture was concentrated under reduced pressure, diluted in water (50 mL) and extracted with AcOEt (30 mL×3). The organic phase was concentrated, and the residue was purified by chromatography column (petroleum ether/AcOEt: 50/1→5/1). The product of interest was obtained in 82.2% yield (5.0 g) in the form of a yellow oil.

### Step V-D-II:

### Precursors V-21II and V-22II:

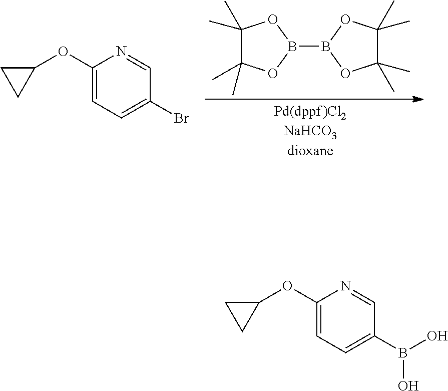

In a round-bottomed flask containing 5-bromo-2-cyclopropoxypyridine (23.4 mmol) in dioxane (50.0 mL), 4,4,5,5-tetramethyl-2-(4,4,5,5-tetramethyl-1,3,2-dioxaborolan-2-yl)-1,3,2-dioxaborolan (28.0 mmol), KOAc (70.1 mmol), and Pd(dppf)Cl₂ (2.34 mmol) were added. The mixture was heated to 90° C. and kept under agitation for 16 hours in an inert atmosphere. After total consumption of the starting reagent (monitored by LC-MS), the mixture was concentrated under reduced pressure, diluted in water (50 mL) and extracted with AcOEt (30 mL×3). The organic phase was concentrated, and the residue was purified by chromatography column (petroleum ether/AcOEt: 50/1-5/1). The product of interest was obtained at 80.4% yield (5.6 g) in the form of a white solid.

### Step V-D-III:

### V-21 and V-22 Intermediates:

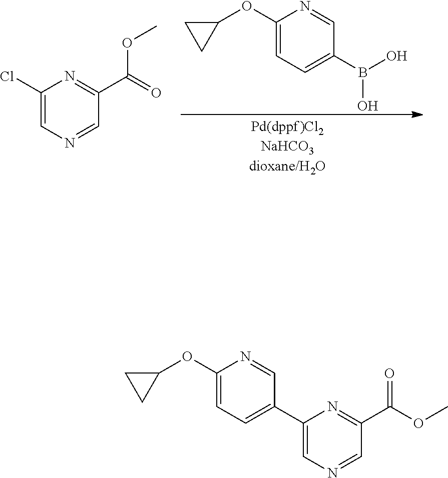

In a round-bottomed flask containing methyl 6-chloropyrazine-2-carboxylate (31.3 mmol) in dioxane (50.0 mL) and water (10 mL), (6-cyclopropoxypyridin-3-yl)boronic acid (37.5 mmol), NaHCO₃ (62.6 mmol) and Pd(dppf)Cl₂ (1.6 mmol) were added. The mixture was heated to 60° C. and kept under agitation for 12 hours in an atmosphere of N₂. After total consumption of the starting reagent (monitored by LC-MS), the mixture was concentrated under reduced pressure, diluted in water (50 mL) and extracted with AcOEt (30 mL×3). The organic phase was concentrated, and the residue was purified by chromatography column (petroleum ether/AcOEt: 30/1-2/1). 5.0 g of the product of interest (49.5% yield) were obtained in the form of a yellow oil.

TABLE 2
INTERMEDIATES OBTAINED FROM THE CORRESPONDING
REAGENTS FOLLOWING STEP V-D-III.

| Intermediate | Structure |
|---|---|
| V-21 | 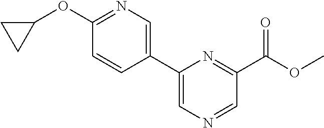 [SMILES: COC(=O)c1cncc(-c2ccc(OC3CC3)nc2)n1] |
| V-22 | 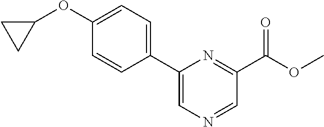 [SMILES: COC(=O)c1cncc(-c2ccc(OC3CC3)cc2)n1] |

### V-E Method

### Step V-E-I:

### Precursor V-22I:

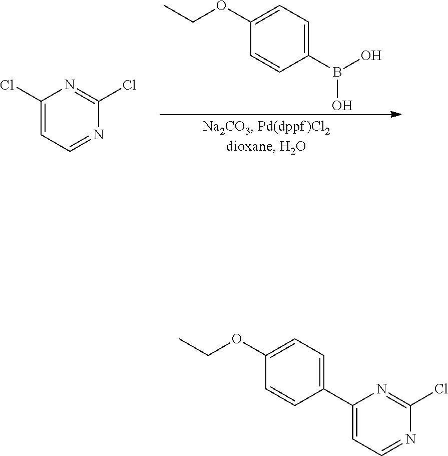 [SMILES: Clc1ccnc(Cl)n1 ; CCOc1ccc(B(O)O)cc1 ; CCOc1ccc(-c2ccnc(Cl)n2)cc1]

In a round-bottomed flask containing 2,4-dichloropyrimidinemethyl (67.1 mmol) in dioxane (100 mL) and water (30 mL), (4-ethoxyphenyl)boronic acid (67.1 mmol), NaHCO₃ (201 mmol) and Pd(dppf)Cl₂ (671 μmol) were added. The mixture was heated to 60° C. and kept under agitation for 16 hours in an atmosphere of N₂. After total consumption of the starting reagent (monitored by LC-MS), the mixture was concentrated under reduced pressure, diluted in water (150 mL) and extracted with AcOEt (50 mL×3). The organic phase was concentrated, and the residue was purified by chromatography column (petroleum ether/AcOEt: 20/1→1/1). 8.9 g of the product of interest (56.5% yield) were obtained in the form of a white solid.

### Step V-E-II:

### Intermediate V-22:

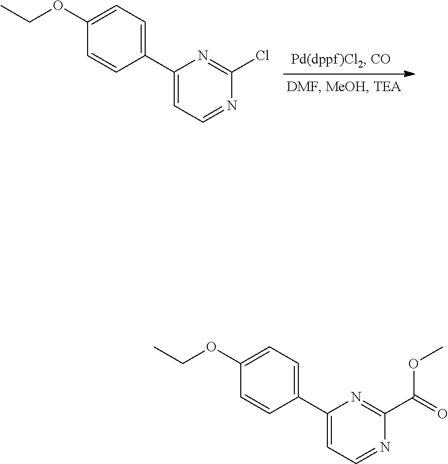 [SMILES: CCOc1ccc(-c2ccnc(Cl)n2)cc1 ; CCOc1ccc(-c2ccnc(C(=O)OC)n2)cc1]

In a round-bottomed flask containing 2-chloro-4-(4-ethoxyphenyl)pyrimidine (21.3 mmol) in methanol (25 mL) and DMF (25 mL), triethylamine (25 mL) and Pd(dppf)Cl₂ (1.07 mmol) were added. The mixture was heated to 80° C., CO (50 Psi) was applied and kept under agitation for 16 hours. After total consumption of the starting reagent (monitored by LC-MS), the mixture was concentrated under reduced pressure, diluted in water (80 mL) and extracted with AcOEt (20 mL×3). The organic phase was concentrated, and the residue was purified by chromatography column (petroleum ether/AcOEt: 20/1-1/1). 4.45 g of the product of interest (80.9% yield) were obtained in the form of a brown solid.

### Formula IV Intermediates:

Following General Scheme 1, the intermediates of Formula IV are presented, however, such methods are not limiting.

### Method IV-A

### Intermediates IV-1 to IV-21:

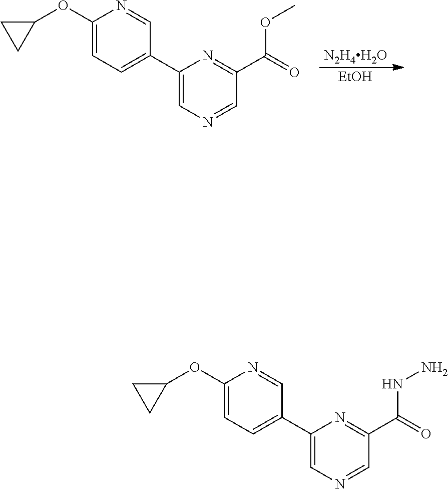

In a round-bottomed flask containing methyl 6-(6-cyclopropoxypyridin-3-yl)pyrazine-2-carboxylate (18.4 mmol) in EtOH (35.0 mL) N₂H₄-H₂O (56.7 mmol) was added. The reaction mixture was kept under agitation at 90° C. for 12 hours. After total consumption of the starting reagent (monitored by CCD), the mixture was filtered and concentrated. 3.80 g (76% yield) of the hydrazide of interest were obtained in the form of a white solid, which was sent to the next step without further purification.

TABLE 3
INTERMEDIATES OBTAINED FROM THE CORRESPONDING
REAGENTS FOLLOWING METHOD IV-A.

| Intermediate | Structure |
|---|---|
| IV-1 | 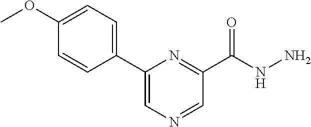 [SMILES: COc1ccc(-c2cncc(C(=O)NN)n2)cc1] |
| IV-2 | 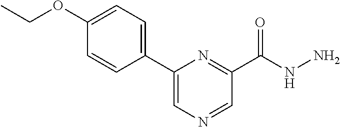 [SMILES: CCOc1ccc(-c2cncc(C(=O)NN)n2)cc1] |
| IV-3 | 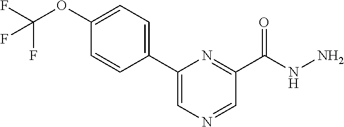 [SMILES: NNC(=O)c1cncc(-c2ccc(OC(F)(F)F)cc2)n1] |
| IV-4 | 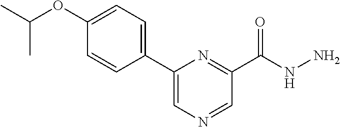 [SMILES: CC(C)Oc1ccc(-c2cncc(C(=O)NN)n2)cc1] |
| IV-5 | 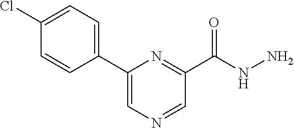 [SMILES: NNC(=O)c1cncc(-c2ccc(Cl)cc2)n1] |
| IV-6 | 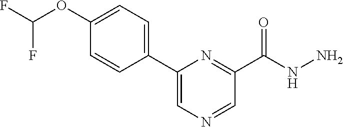 [SMILES: NNC(=O)c1cncc(-c2ccc(OC(F)F)cc2)n1] |
| IV-7 | 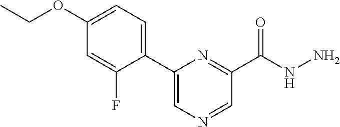 [SMILES: CCOc1ccc(-c2cncc(C(=O)NN)n2)c(F)c1] |
| IV-8 | 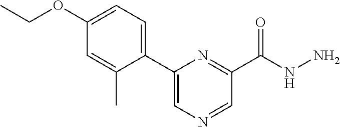 [SMILES: CCOc1ccc(-c2cncc(C(=O)NN)n2)c(C)c1] |
| IV-9 | 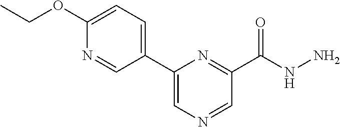 [SMILES: CCOc1ccc(-c2cncc(C(=O)NN)n2)cn1] |
| IV-10 | 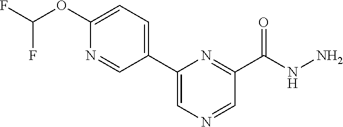 [SMILES: NNC(=O)c1cncc(-c2ccc(OC(F)F)nc2)n1] |
| IV-11 | 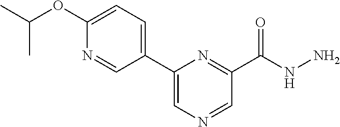 [SMILES: CC(C)Oc1ccc(-c2cncc(C(=O)NN)n2)cn1] |
| IV-12 | 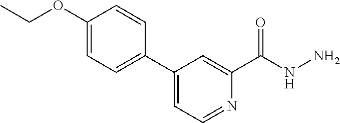 [SMILES: CCOc1ccc(-c2ccnc(C(=O)NN)c2)cc1] |
| IV-13 | 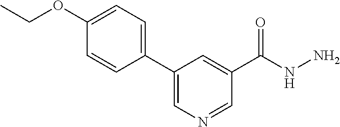 [SMILES: CCOc1ccc(-c2cncc(C(=O)NN)c2)cc1] |
| IV-14 | 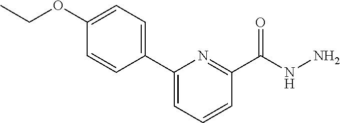 [SMILES: CCOc1ccc(-c2cccc(C(=O)NN)n2)cc1] |
| IV-15 | 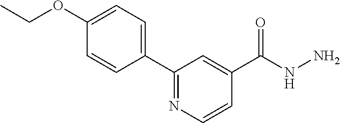 [SMILES: CCOc1ccc(-c2cc(C(=O)NN)ccn2)cc1] |
| IV-16 | 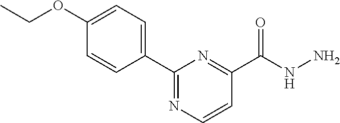 [SMILES: CCOc1ccc(-c2nccc(C(=O)NN)n2)cc1] |
| IV-17 | 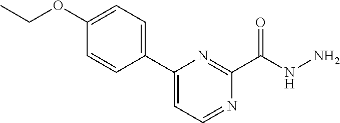 [SMILES: CCOc1ccc(-c2ccnc(C(=O)NN)n2)cc1] |
| IV-18 | 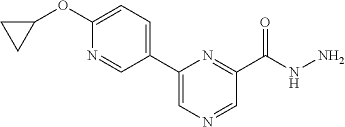 [SMILES: NNC(=O)c1cncc(-c2ccc(OC3CC3)nc2)n1] |
| IV-19 | 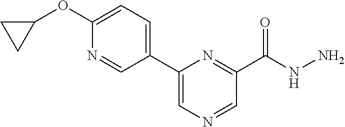 [SMILES: NNC(=O)c1cncc(-c2ccc(OC3CC3)nc2)n1] |
| IV-20 | 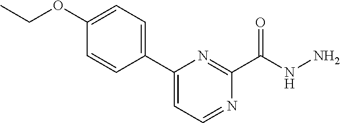 [SMILES: CCOc1ccc(-c2ccnc(C(=O)NN)n2)cc1] |
| IV-21 | 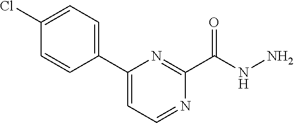 [SMILES: NNC(=O)c1nccc(-c2ccc(Cl)cc2)n1] |

### Method IV-B

### Step IV-B-I:

### Precursors IV-22I and IV-23I:

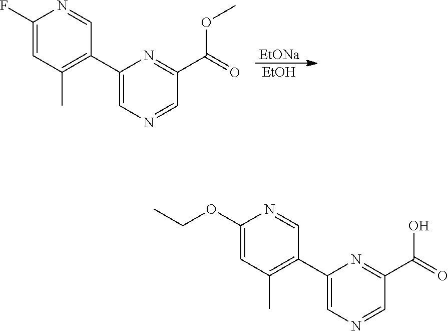 [SMILES: "EtONa" ; "EtOH" ; COC(=O)c1cncc(-c2cnc(F)cc2C)n1 ; CCOc1cc(C)c(-c2cncc(C(=O)O)n2)cn1]

In a round-bottomed flask containing methyl 6-(6-fluoro-4-methylpyridin-3-yl)pyrazine-2-carboxylate (19.4 mmol) in EtOH (50.0 mL), EtONa (135.9 mmol) was added. The reaction mixture was kept under agitation at 90° C. for 16 hours. After total consumption of the starting reagent (monitored by LC-MS), the mixture was concentrated under reduced pressure, diluted in water (50 mL) and extracted with AcOEt (30 mL×3). The organic phase was concentrated and the residue obtained was used directly in the next step without further purification. 2.5 g of the product of interest (24.8% yield) were obtained in the form of a yellow solid.

TABLE 4
PRECURSORS OBTAINED FROM THE CORRESPONDING
REAGENTS FOLLOWING STEP IV-B-I.

| Precursor | Structure |
|---|---|
| IV-22i | 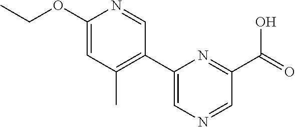 [SMILES: CCOc1cc(C)c(-c2cncc(C(=O)O)n2)cn1] |
| IV-22i | 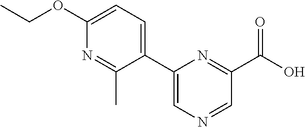 [SMILES: CCOc1ccc(-c2cncc(C(=O)O)n2)c(C)n1] |

### Step IV-B-II:

### Precursors IV-22II and IV-23II:

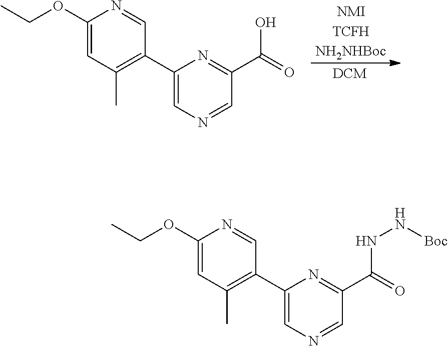

In a round-bottomed flask containing 6-(6-ethoxy-4-methylpyridin-3-yl)pyrazine-2-carboxylic acid (4.82 mmol) in DCM (20.0 mL) tert-butylN-aminocarbamate (5.06 mmol), TCFH (7.23 mmol), and NMI (9.64 mmol) were added. The reaction mixture was kept under agitation at room temperature for 3 hours. After total consumption of the starting reagent (monitored by LC-MS), the mixture was concentrated under reduced pressure, diluted in water (20 mL) and extracted with AcOEt (10 mL×3). The organic phase was concentrated, and the residue was purified by chromatography column (petroleum ether/AcOEt: 30/1→2/1) and by HPLC prep [H₂O(NH₄CO₃)-ACN](gradient 30%-60% B). 3.74 g of the product of interest (77.6% yield) were obtained in the form of a yellow oil.

TABLE 5
PRECURSORS OBTAINED FROM THE CORRESPONDING
REAGENTS FOLLOWING STEP IV-B-II.

| Precursor | Structure |
|---|---|
| IV-22ii | 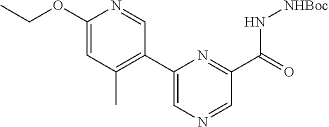 [SMILES: CCOc1cc(C)c(-c2cncc(C(=O)NNC(=O)OC(C)(C)C)n2)cn1] |
| IV-22ii | 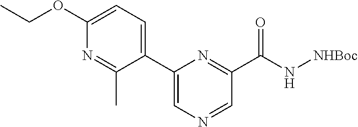 [SMILES: CCOc1ccc(-c2cncc(C(=O)NNC(=O)OC(C)(C)C)n2)c(C)n1] |

### Step IV-B-III:

### Intermediates IV-22 and IV-23:

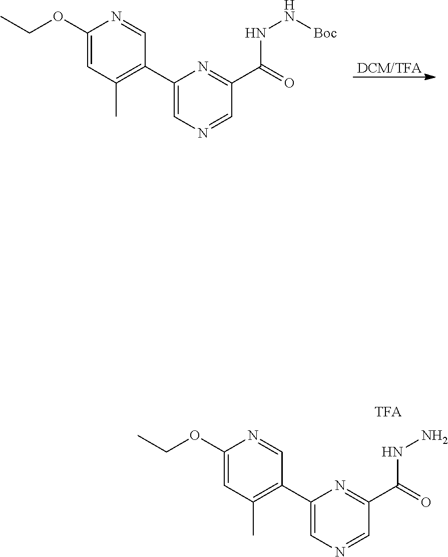 [SMILES: CCOc1cc(C)c(-c2cncc(C(=O)NNC(=O)OC(C)(C)C)n2)cn1 ; "DCM/TFA" ; CCOc1cc(C)c(-c2cncc(C(=O)NN)n2)cn1]

In a round-bottomed flask containing tert-butyl 2-(6-(6-ethoxy-4-methylpyridin-3-yl)pyrazine-2-carbonyl)hydrazine-1-carboxylate (3.75 mmol) in DCM (15.0 mL) TFA (5.0 mL) was added. The reaction mixture was kept under agitation at room temperature for 1 hour. After total consumption of the starting reagent (monitored by CCD), the mixture was concentrated under reduced pressure. 2.2 g of the product of interest (90.9% yield) were obtained in the form of a black solid that was used in the next step without further purification.

TABLE 6
INTERMEDIATES OBTAINED FROM THE CORRESPONDING
REAGENTS FOLLOWING STEP IV-B-III.

| Intermediate | Structure |
|---|---|
| IV-22 | 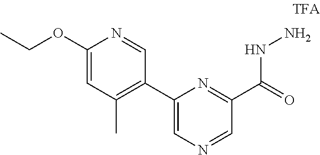 [SMILES: CCOc1cc(C)c(-c2cncc(C(=O)NN)n2)cn1] |
| IV-23 | 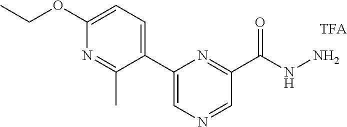 [SMILES: CCOc1ccc(-c2cncc(C(=O)NN)n2)c(C)n1] |

### Formula VI Intermediates:

Following General Scheme 1, Formula VI intermediates are presented, but such methods are not limiting.

### Method VI-A

### Intermediates VI-1 to VI-3:

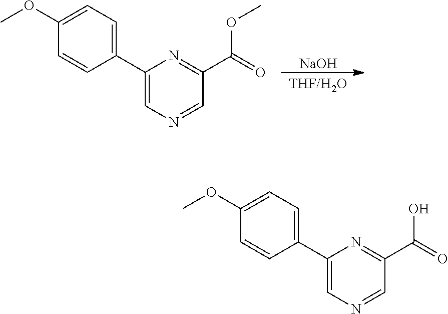

In a round-bottomed flask containing methyl 6-(4-methoxyphenyl)pyrazine-2-carboxylate (15.2 mmol) in THF (50.0 mL), NaOH (22.9 mmol) was added to water (10.0 mL). The reaction mixture was kept under agitation at room temperature for 12 hours. After total consumption of the starting reagent (monitored by LC-MS), the pH was adjusted to 5 with 2N HCL, and the precipitate formed was filtered. The filtrate was concentrated and used directly in the next step without further purification. 3.5 g of the product of interest (97.6% yield) were obtained in the form of a white solid.

TABLE 7
INTERMEDIATES OBTAINED FROM THE CORRESPONDING
REAGENTS FOLLOWING METHOD VI-A.

| Intermediate | Structure |
|---|---|
| IV-1 | 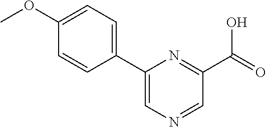 [SMILES: COc1ccc(-c2cncc(C(=O)O)n2)cc1] |
| IV-2 | 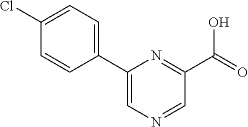 [SMILES: O=C(O)c1cncc(-c2ccc(Cl)cc2)n1] |
| IV-3 | 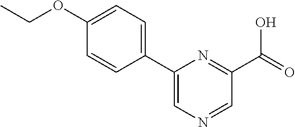 [SMILES: CCOc1ccc(-c2cncc(C(=O)O)n2)cc1] |

### Formula VII Intermediates:

Following General Scheme 1, the intermediates of Formula VII are presented, however, such methods are not limiting.

### Method VII-A

### Intermediate VII-1:

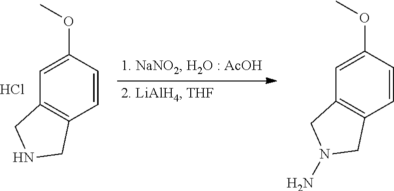

In a round-bottomed flask containing a solution of NaNO₂ (2.00 eq.) in H₂O (30 mL), 5-methoxyisoindoline hydrochloride (1.00 eq., HCl) was added. Next, AcOH (3 mL) was added at 20° C. and the reaction mixture was kept under agitation for 2 hours. After total consumption of the starting reagent (monitored by CCD), the mixture was filtered and the residue obtained was lyophilized. Then, the crude reaction was dissolved in THF (45.0 mL) and LiAlH₄ (2.0 eq.) was added in portions to the reaction balloon. The reaction mixture was kept under agitation at 20° C. for 3 hours. After total consumption of the starting reagent (monitored by CCD and LC-MS), NaNO₂ (2.0 eq.) was added in H₂O, the reactive crude was filtered under celite and the residue obtained was purified by HPLC ([H₂O(NH₄HCO₃)-ACN]; B %: 10%-40%).

### Method VII-B

### Intermediate VII-2:

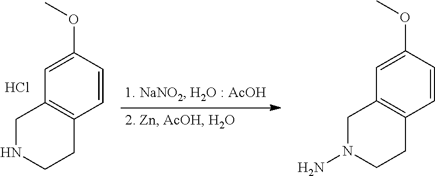

Step 1 was carried out by means of an experimental procedure equivalent to Step 1 of Intermediate VII-1-Method VII-A, through the use of the corresponding starting materials. Then, the crude reaction was dissolved in AcOH (10.0 mL) and H₂O (10.0 mL) and Zn (5.00 eq.) was added in portions to the reaction balloon. The reaction mixture remained at 20° C. for 16 hours. After total consumption of the starting reagent (monitored by CCD and LC-MS), the mixture was filtered under celite and the residue obtained was purified by HPLC ([H₂O(HCl)-ACN]1%→21% B). Next, MeCN was evaporated, and the obtained product was lyophilized.

### Method VII-C

### Step VII-C-I:

### Precursor VII-3I:

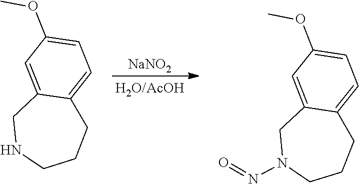

In a round-bottomed flask containing 8-methoxy-2,3,4,5-tetrahydro-1H-benzo[c]azepine (5.64 mmol) in H₂O (10.0 mL) and AcOH (10.0 mL), NaNO₂ (16.9 mmol) was added. The reaction mixture remained at 0° C. for 3 hours. After total consumption of the starting reagent (monitored by LC-MS), the pH of the mixture was adjusted to 8 by adding an aqueous solution of Na₂CO₃. The residue obtained was diluted with H₂O (20.0 mL) and extracted with AcOEt (20.0 mL×3). The organic phase was concentrated under reduced pressure. 1.00 g (85.9% yield) of the product of interest were obtained in the form of a yellow oil, which was used for the next step without further purification.

### Step VII-C-II:

### Intermediate VII-3:

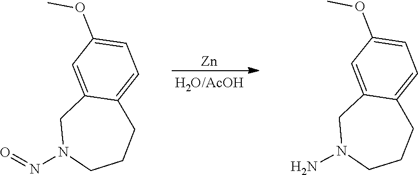

In a round-bottomed flask containing 8-methoxy-2-nitrous-2,3,4,5-tetrahydro-1H-benzo[c]azepine (4.85 mmol) in AcOH (10.0 mL) and H₂O (10.0 mL), Zn (24.2 mmol) was added in portions at 0° C. The reaction mixture remained at 20° C. for 16 hours. After total consumption of the starting reagent (monitored by CCD and LC-MS), the mixture was filtered, and the pH of the organic phase was adjusted to 8 by adding an aqueous solution of Na₂CO₃. The residue obtained was diluted with H₂O (20.0 mL) and extracted with AcOEt (10.0 mL×3). The organic phase was concentrated under reduced pressure. 700 mg (75.0% yield) of the product of interest were obtained in the form of a yellow oil, which was used for the next step without further purification.

### Method VII-D

### Step VII-D-I:

### Precursor VII-4I:

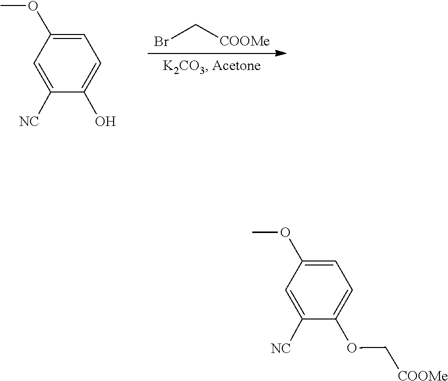 [SMILES: COc1ccc(O)c(C#N)c1 ; COC(=O)CBr ; COC(=O)COc1ccc(OC)cc1C#N]

In a round-bottomed flask containing 2-hydroxy-5-methoxybenzonitrile (33.5 mmol) and K₂CO₃ (50.2 mmol) in acetone (50.0 mL), methyl 2-bromoacetate (36.8 mmol) was added dropwise at 20° C. The reaction mixture remained at 20° C. for 16 hours. After total consumption of the starting reagent (monitored by CCD and LC-MS), the mixture was diluted with H₂O (100 mL) and extracted with AcOEt (50.0 mL×3). The organic phase was concentrated under reduced pressure. 7.40 g (99.7% yield) of the product of interest were obtained in the form of a white solid, which was used for the next step without further purification.

### Step VII-D-II:

### Precursor VII-4II:

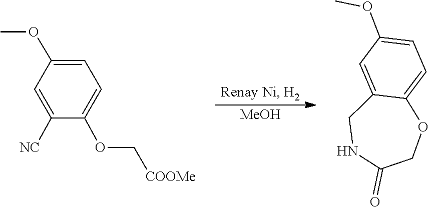 [SMILES: "MeOH" ; COC(=O)COc1ccc(OC)cc1C#N ; COc1ccc2c(c1)CNC(=O)CO2]

In a round-bottomed flask containing 2-(2-cyano-4-methoxyphenoxy)methyl acetate (33.4 mmol) in MeOH (70.0 mL), Raney-Ni (172 mmol) and NH₃·H₂O (33.4 mmol) was added under the atmosphere of N₂. The suspension was de-gassed and purged with H₂. The mixture was kept in agitation under an atmosphere of H₂ (50 psi) at 50° C. for 6 hours. After total consumption of the starting reagent (monitored by LC-MS), the mixture was filtered, washed with MeOH (50.0 mL×3) and concentrated. 6.00 g (92.8% yield) of the product of interest were obtained in the form of a white solid, which was used for the next step without further purification.

### Step VII-D-III:

### Precursor VII-4III:

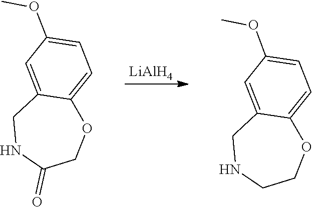

In a round-bottomed flask containing 7-methoxy-4,5-dihydrobenzo [1,4]oxazepine-3 (2H)-one (10.3 mmol) in THF (20 mL), LiAlH₄ (41.4 mmol) was added at 0° C. Subsequently, the reaction mixture was kept in agitation at 70° C. for 12 hours. After total consumption of the starting reagent (monitored by LC-MS), H₂O (2.0 mL) and NaOH aqueous solution (2.0 mL) were added to the reaction mixture. Next, the mixture was diluted with H₂O (10.0 mL) and extracted with AcOEt (10.0 mL×3). The organic phase was concentrated under reduced pressure. 1.80 g (97.02% yield) of the product of interest were obtained in the form of a yellow solid, which was used for the next step without further purification.

### Step VII-D-IV:

### Precursor VII-4IV:

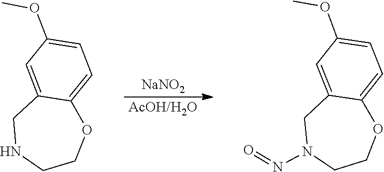

In a round-bottomed flask containing 7-methoxy-2,3,4,5-tetrahydrobenzo[1,4]oxazepine (10.0 mmol) in AcOH (10.0 mL), NaNO₂ (30.1 mmol) was added. The reaction mixture remained at 0° C. for 3 hours. After total consumption of the starting reagent (monitored by LC-MS), the pH of the mixture was adjusted to 8 by adding an aqueous solution of Na₂CO₃. The residue obtained was diluted with H₂O (50.0 mL) and extracted with AcOEt (30.0 mL×3). The organic phase was concentrated under reduced pressure. 1.80 g (86.0% yield) of the product of interest were obtained in the form of a yellow solid, which was used for the next step without further purification.

### Step VII-D-V:

### Intermediate VII-4:

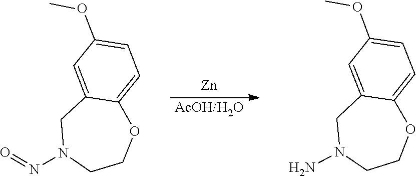

In a round-bottomed flask containing 7-methoxy-4-nitrous-2,3,4,5-tetrahydrobenzo[1,4]oxazepine (8.64 mmol) in AcOH (10.0 mL) and H₂O (10.0 mL), Zn (48.1 mmol) was added in portions at 0° C. The reaction mixture remained at 20° C. for 16 hours. After total consumption of the starting reagent (monitored by LC-MS), the mixture was filtered, and the pH of the organic phase was adjusted to 8 by adding an aqueous solution of Na₂CO₃. The residue obtained was diluted with H₂O (20.0 mL) and extracted with AcOEt (20.0 mL×3). The organic phase was concentrated under reduced pressure. 700 mg (41.6% yield) of the product of interest were obtained in the form of a yellow oil, which was used for the next step without further purification.

### Formula II Intermediates:

Following General Scheme 1, the Formula II intermediates are presented, however such methods are not exhaustive.

### Method II-A

### Intermediates II-1 to II-23:

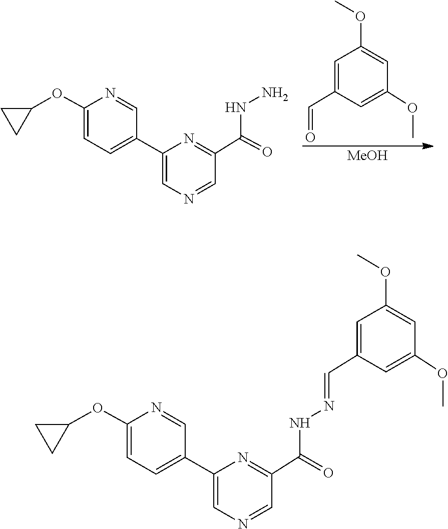

In a round-bottomed flask containing 6-(6-cyclopropoxypyridin-3-yl)pyrazine-2-carbohydrazide (1.11 mmol) in methanol (3 mL) 3,5-dimethoxybenzaldehyde (1.16 mmol) was added. The reaction mixture was kept under agitation at 60° C. for 12 hours. Upon conclusion of the reaction, the mixture was filtered, the residue washed with MeOH (5 mL) and the filtrate was concentrated under reduced pressure. 400 mg of the product of interest (86.1% yield) were obtained in the form of a white solid that was used for the next step without further purification.

TABLE 8
INTERMEDIATES OBTAINED FROM THE CORRESPONDING
REAGENTS FOLLOWING METHOD IIA.

| Intermediate | Structure |
|---|---|
| II-1 | 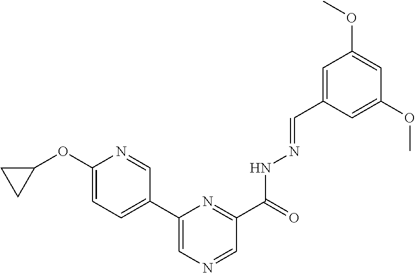 [SMILES: COc1cc(/C=N/NC(=O)c2cncc(-c3ccc(OC4CC4)nc3)n2)cc(OC)c1] |
| II-2 | 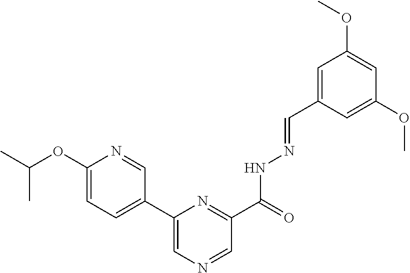 [SMILES: COc1cc(/C=N/NC(=O)c2cncc(-c3ccc(OC(C)C)nc3)n2)cc(OC)c1] |
| II-3 | 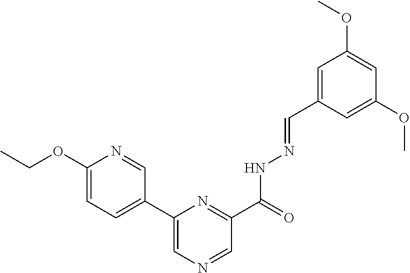 [SMILES: CCOc1ccc(-c2cncc(C(=O)N/N=C/c3cc(OC)cc(OC)c3)n2)cn1] |
| II-4 | 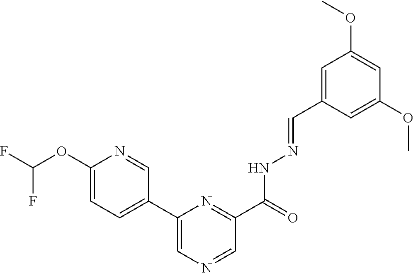 [SMILES: COc1cc(/C=N/NC(=O)c2cncc(-c3ccc(OC(F)F)nc3)n2)cc(OC)c1] |
| II-5 | 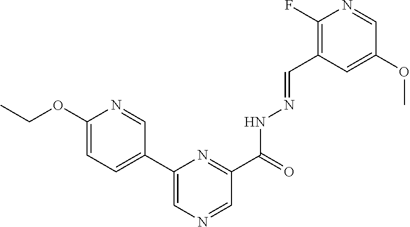 [SMILES: CCOc1ccc(-c2cncc(C(=O)N/N=C/c3cc(OC)cnc3F)n2)cn1] |
| II-6 | 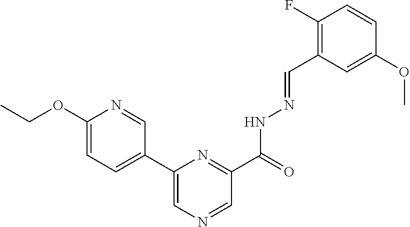 [SMILES: CCOc1ccc(-c2cncc(C(=O)N/N=C/c3cc(OC)ccc3F)n2)cn1] |
| II-7 | 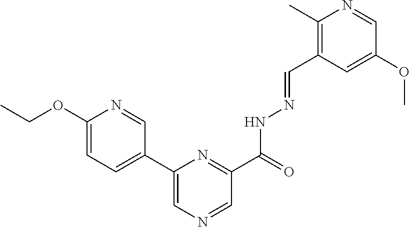 [SMILES: CCOc1ccc(-c2cncc(C(=O)N/N=C/c3cc(OC)cnc3C)n2)cn1] |
| II-8 | 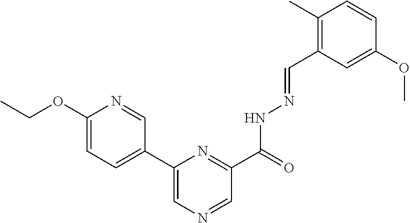 [SMILES: CCOc1ccc(-c2cncc(C(=O)N/N=C/c3cc(OC)ccc3C)n2)cn1] |
| II-9 | 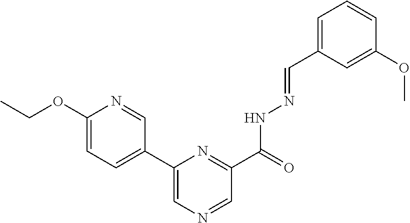 [SMILES: CCOc1ccc(-c2cncc(C(=O)N/N=C/c3cccc(OC)c3)n2)cn1] |
| II-10 | 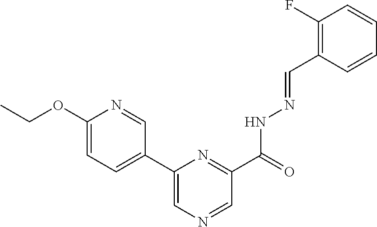 [SMILES: CCOc1ccc(-c2cncc(C(=O)N/N=C/c3ccccc3F)n2)cn1] |
| II-11 | 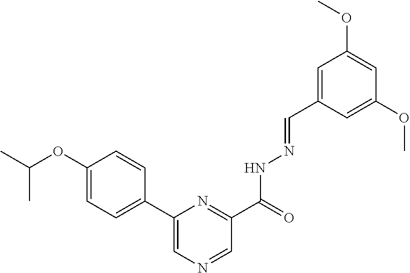 [SMILES: COc1cc(/C=N/NC(=O)c2cncc(-c3ccc(OC(C)C)cc3)n2)cc(OC)c1] |
| II-12 | 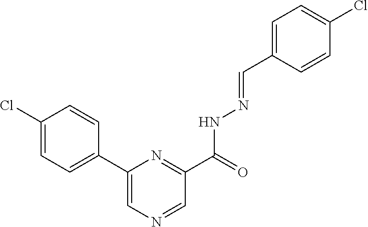 [SMILES: O=C(N/N=C/c1ccc(Cl)cc1)c1cncc(-c2ccc(Cl)cc2)n1] |
| II-12 | 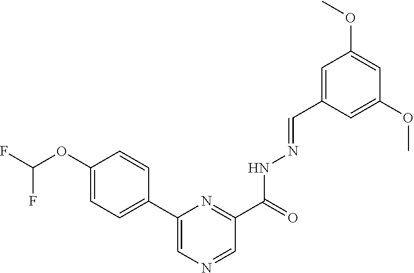 [SMILES: COc1cc(/C=N/NC(=O)c2cncc(-c3ccc(OC(F)F)cc3)n2)cc(OC)c1] |
| II-13 | 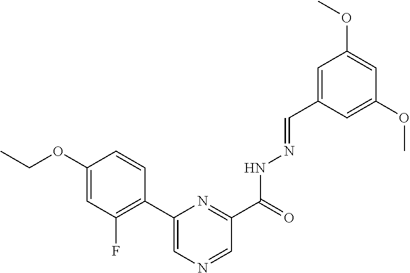 [SMILES: CCOc1ccc(-c2cncc(C(=O)N/N=C/c3cc(OC)cc(OC)c3)n2)c(F)c1] |
| II-14 | 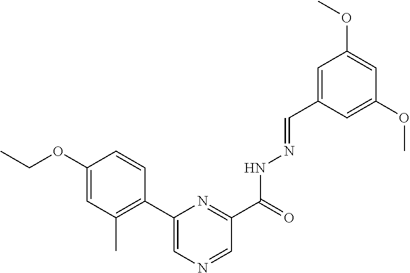 [SMILES: CCOc1ccc(-c2cncc(C(=O)N/N=C/c3cc(OC)cc(OC)c3)n2)c(C)c1] |
| II-15 | 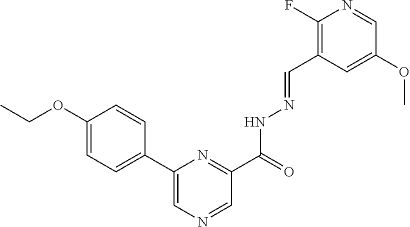 [SMILES: CCOc1ccc(-c2cncc(C(=O)N/N=C/c3cc(OC)cnc3F)n2)cc1] |
| II-16 | 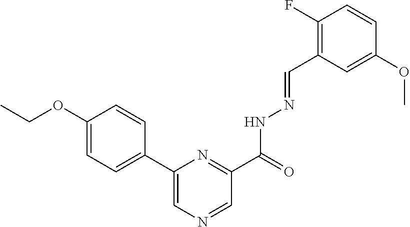 [SMILES: CCOc1ccc(-c2cncc(C(=O)N/N=C/c3cc(OC)ccc3F)n2)cc1] |
| II-17 | 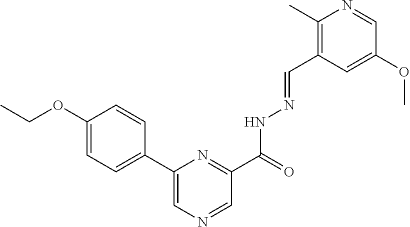 [SMILES: CCOc1ccc(-c2cncc(C(=O)N/N=C/c3cc(OC)cnc3C)n2)cc1] |
| II-18 | 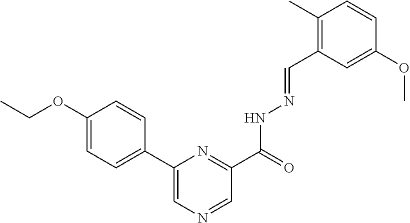 [SMILES: CCOc1ccc(-c2cncc(C(=O)N/N=C/c3cc(OC)ccc3C)n2)cc1] |
| II-19 | 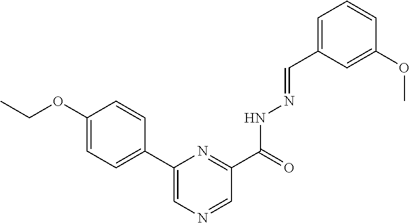 [SMILES: CCOc1ccc(-c2cncc(C(=O)N/N=C/c3cccc(OC)c3)n2)cc1] |
| II-20 | 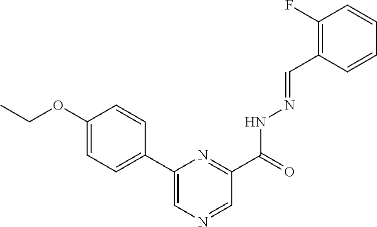 [SMILES: CCOc1ccc(-c2cncc(C(=O)N/N=C/c3ccccc3F)n2)cc1] |
| II-21 | 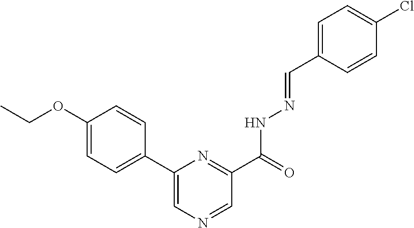 [SMILES: CCOc1ccc(-c2cncc(C(=O)N/N=C/c3ccc(Cl)cc3)n2)cc1] |
| II-22 | 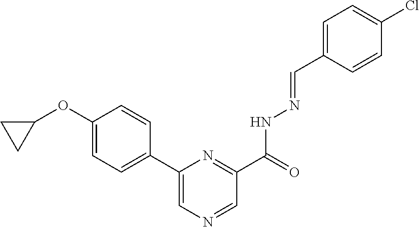 [SMILES: O=C(N/N=C/c1ccc(Cl)cc1)c1cncc(-c2ccc(OC3CC3)cc2)n1] |
| II-23 | 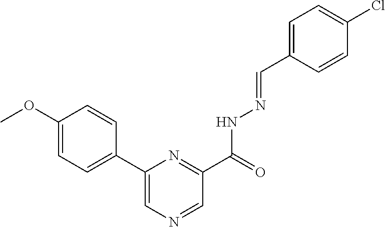 [SMILES: COc1ccc(-c2cncc(C(=O)N/N=C/c3ccc(Cl)cc3)n2)cc1] |

### Method II-B

### Intermediates II-24 and II-25:

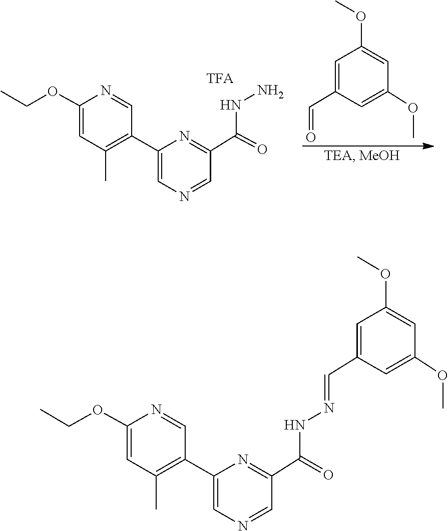

In a round-bottomed flask containing 6-(6-ethoxy-4-methylpyridin-3-yl)pyrazine-2-carbohydrazide (3.41 mmol) in methanol (20 mL), TEA (3.41 mmol) and 3,5-dimethoxybenzaldehyde (3.75 mmol) were added. The reaction mixture was kept under agitation at 50° C. for 12 hours. Upon conclusion of the reaction, the mixture was filtered, the residue washed with MeOH (5 mL) and the filtrate was concentrated under reduced pressure. 1.2 g of the product of interest (80.2% yield) were obtained in the form of a white solid that was used for the next step without further purification.

TABLE 9
INTERMEDIATES OBTAINED FROM THE CORRESPONDING
REAGENTS FOLLOWING METHOD II-B.

| Intermediate | Structure |
|---|---|
| II-24 | 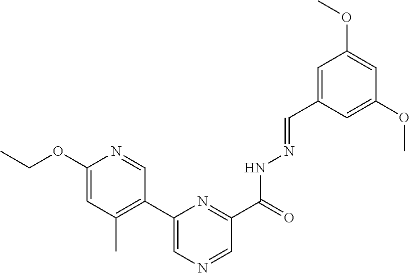 [SMILES: CCOc1cc(C)c(-c2cncc(C(=O)N/N=C/c3cc(OC)cc(OC)c3)n2)cn1] |
| II-25 | 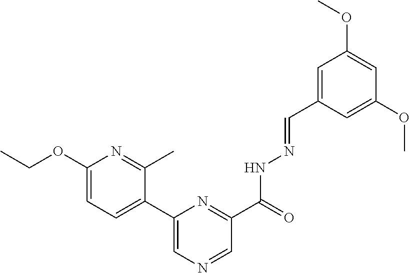 [SMILES: CCOc1ccc(-c2cncc(C(=O)N/N=C/c3cc(OC)cc(OC)c3)n2)c(C)n1] |

### Method II-C

### Intermediate II-26:

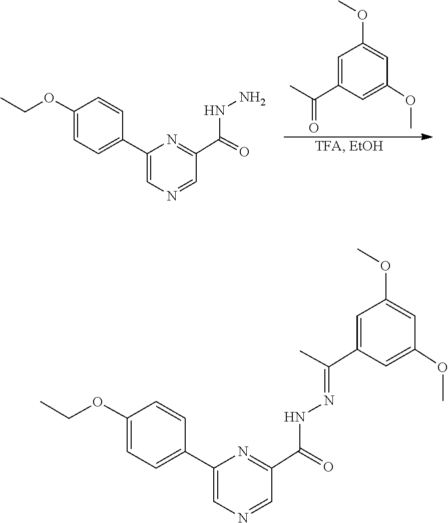

In a round-bottomed flask containing 6-(4-ethoxyphenyl)pyrazine-2-carbohydrazide (3.87 mmol) in ethanol (15 mL), TFA (7.74 mmol), 1-(3,5-dimethoxyphenyl)ethane (4.07 mmol) and 4A molecular sieve (3.87 mmol) were added. The reaction mixture was kept under agitation at 90° C. for 16 hours. Upon conclusion of the reaction, the mixture was diluted in DCM (20 mL), the precipitate formed was filtered and washed with DCM and the resulting filtrate was concentrated under reduced pressure. 1.55 g of the product of interest (95.2% yield) were obtained in the form of a white solid that was used for the next step without further purification.

### Method II-D

### Intermediates II-27 to II-32:

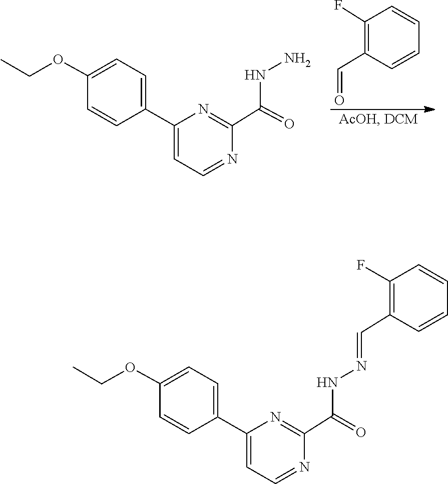

In a round-bottomed flask containing 4-(4-ethoxyphenyl)pyrimidine-2-carbohydrazide (3.10 mmol) and 2-fluorobenzaldehyde (3.25 mmol) in DCM (8.0 mL) AcOH (3.10 mmol) was added. The reaction mixture was kept under agitation at room temperature for 1 hour. Upon conclusion of the reaction, the mixture was concentrated and the residue obtained was crushed with MTBE (5.0 mL). 1.1 g of the product of interest (97.5% yield) were obtained in the form of a white solid.

TABLE 10
INTERMEDIATES OBTAINED FROM THE
CORRESPONDING REAGENTS FOLLOWING METHOD II-D.

| Intermediate | Structure |
|---|---|
| II-27 | 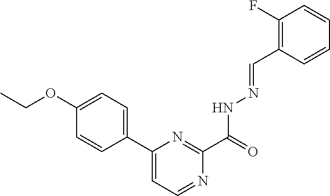 [SMILES: CCOc1ccc(-c2ccnc(C(=O)N/N=C/c3ccccc3F)n2)cc1] |
| II-28 | 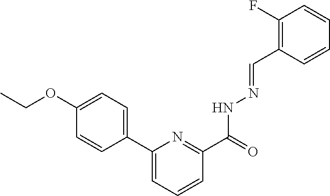 [SMILES: CCOc1ccc(-c2cccc(C(=O)N/N=C/c3ccccc3F)n2)cc1] |
| II-29 | 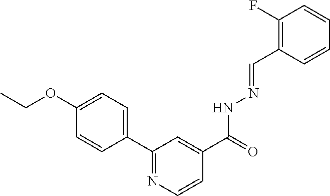 [SMILES: CCOc1ccc(-c2cc(C(=O)N/N=C/c3ccccc3F)ccn2)cc1] |
| II-30 | 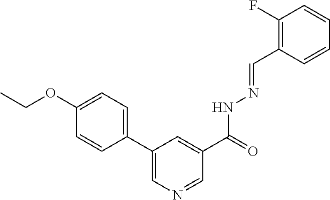 [SMILES: CCOc1ccc(-c2cncc(C(=O)N/N=C/c3ccccc3F)c2)cc1] |
| II-31 | 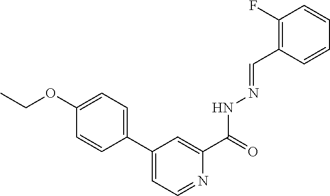 [SMILES: CCOc1ccc(-c2ccnc(C(=O)N/N=C/c3ccccc3F)c2)cc1] |
| II-32 | 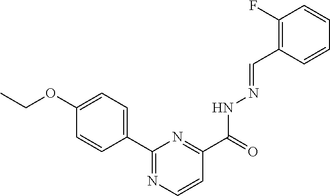 [SMILES: CCOc1ccc(-c2nccc(C(=O)N/N=C/c3ccccc3F)n2)cc1] |

### EXAMPLES

The following examples, described in detail, serve to illustrate the embodiments of this invention without, however, having any limitation character to the scope of protection thereof.

The compound(s) of General Formula I of this invention were obtained from the reduction of Formula II intermediates using or not acid catalysis and reducing agents, as described above. The preparation methodologies for intermediates II-V are outlined in General Scheme 1, however they are not limited thereto.

### Example 1. 6-(6-Cyclopropoxypyridin-3-il)-N′-(3,5-Dimethoxybenzyl)Pyrazine-2-Carbohydrazide

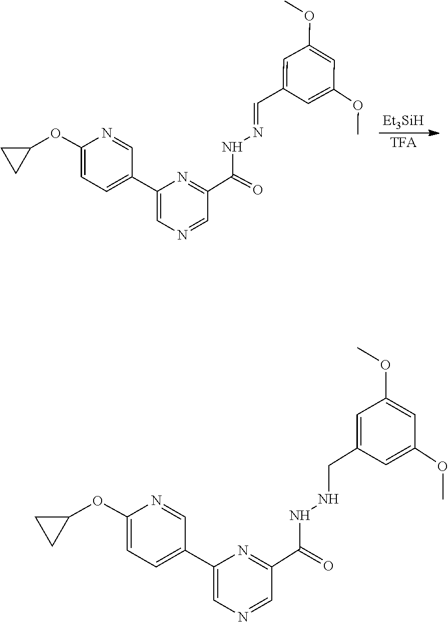 [SMILES: "Et3SiH" ; COc1cc(/C=N/NC(=O)c2cncc(-c3ccc(OC4CC4)nc3)n2)cc(OC)c1 ; COc1cc(CNNC(=O)c2cncc(-c3ccc(OC4CC4)nc3)n2)cc(OC)c1 ; "TFA"]

In a round-bottomed flask containing (E)-6-(6-cyclopropoxypyridin-3-yl)-N′-(3,5-dimethoxybenzylidene)pyrazine-2-carbohydrazide (953.7 μmol) in TFA (4.0 mL), Et₃SiH (1.91 mmol) was added. The reaction mixture was kept at room temperature for 16 hours. After total consumption of the starting reagent (monitored by CCD), the mixture was concentrated under reduced pressure, the residue was diluted in H₂O (10 mL), the pH was adjusted to 8 by adding an aqueous solution of Na₂CO₃, and the mixture was extracted with AcOEt (5.0 mL×3). The organic phase was concentrated under reduced pressure. The residue obtained was purified by prep-TLC (petroleum ether/AcOEt: 20/1→2/1). 260 mg (64.6% yield) of the product of interest were obtained in the form of a yellow solid.

Compounds 1-5, 7-10, 14-16, 22, 24-33, 39-44, 49 and 51-53, as shown in Table 11, are obtained by the procedure described for Example 1, changing the corresponding intermediates II and III.

### Example 2. 6-(4-Ethoxyphenyl)-N′-((5-Methoxy-2-Methylpyridin-3-yl)Methyl)Pyrazine-2-Carbohydrazide

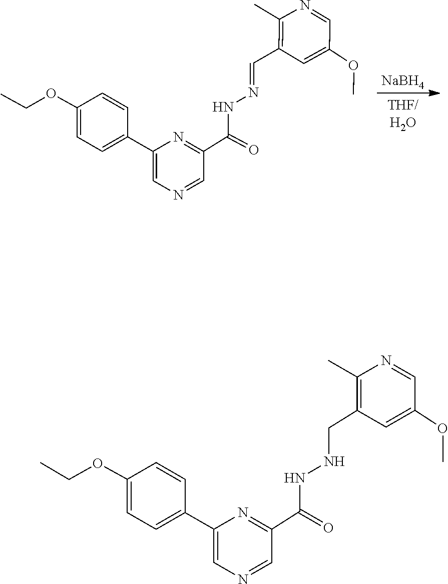

In a round-bottomed flask containing (E)-6-(4-ethoxyphenyl)-N′-((5-methoxy-2-methylpyridin-3-yl)methylene)pyrazine-2-carbohydrazide (1.15 mmol) in THF (50.0 mL) and H₂O (10.0 mL), NaBH₄ (6.34 mmol) was added. The reaction mixture was kept at room temperature for 16 hours. After total consumption of the starting reagent (monitored by CCD), the mixture was cooled to 0° C. and the pH was adjusted to 8 by adding HCL solution (2N). The formed precipitate was filtered and crushed with DCM/methanol (10/1). 200 mg (42.7% yield) of the product of interest were obtained in the form of a yellow solid.

Compounds 18, 34, 48, 50, as shown in Table 11, are obtained by the procedure described for Example 1, changing the corresponding intermediates II and III.

### Example 3. 6-(4-Ethoxyphenyl)-N′-(2-Fluorobenzyl)Pyridazine-4-Carbohydrazide

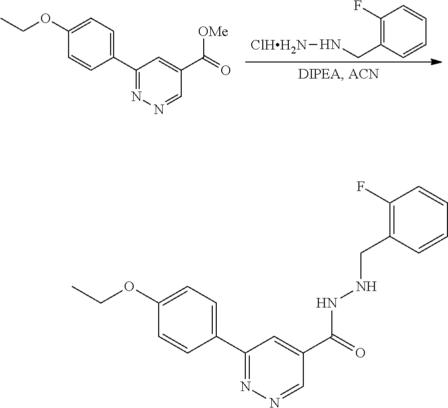

In a round-bottomed flask containing methyl 6-(4-ethoxyphenyl)pyridazine-4-carboxylate (3.87 mmol) and (2-fluorobenzyl)hydrazine hydrochloride (4.64 mmol) in ACN (10.0 mL) and H₂O (10.0 mL), DIPEA (5.0 mL) was added. The reaction mixture remained at 100° C. for 16 hours. After total consumption of the starting reagent (monitored by CCD), the mixture was diluted in water (25 mL) and extracted with DCM (25 mL×3). The organic phase was concentrated under reduced pressure and the residue was purified by prep-HPLC. 160 mg (11.2% yield) of the product of interest were obtained in the form of a white solid.

Compounds 46 and 47, as shown in Table 11, are obtained by the procedure described for Example 1, changing the corresponding intermediates II and III.

### Example 4. N-(6-Chloro-3,4-Dihydroisoquinoline-2 (1H)-il)-6-(4-Chlorophenyl)Pyrazine-2-Carboxamide

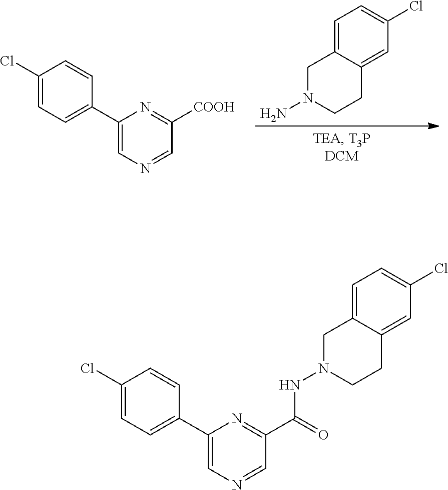 [SMILES: O=C(O)c1cncc(-c2ccc(Cl)cc2)n1 ; NN1CCc2cc(Cl)ccc2C1 ; O=C(NN1CCc2cc(Cl)ccc2C1)c1cncc(-c2ccc(Cl)cc2)n1]

In a round-bottomed flask containing 6-(4-chlorophenyl)pyrazine-2-carboxylic acid (2.56 mmol) in DCM (6.0 mL), T₃P (3.84 mmol), 6-chloro-3,4-dihydroisoquinoline-2 (1H)-amine (2.81 mmol) and TEA (5.11 mmol) were added at 0° C. The reaction mixture was kept at room temperature for 12 hours. After total consumption of the starting reagent (monitored by CCD), the mixture was diluted in water (10 mL) and extracted with DCM (10 mL×2). The organic phase was concentrated under reduced pressure and the residue was purified by prep-HPLC ([H₂O(NH₄HCO₃)-ACN]; B %: 35%-70%). 260 mg (25.4% yield) of the product of interest were obtained in the form of a white solid.

Compounds 6, 11-13, 45 and 47, as shown in Table 11, are obtained by the procedure described for Example 1, changing the corresponding intermediates II and III.

### Example 5. N′-(3,5-Dimethoxybenzyl)-6-(4-Ethoxyphenyl)-N′-Methylpyrazine-2-Carbohydrazide

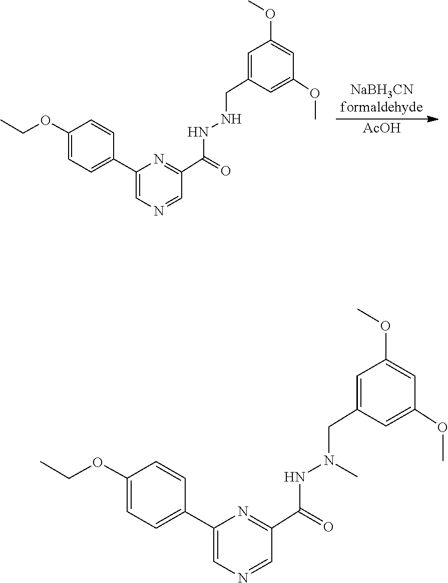

In a round-bottomed flask containing N′-(3,5-dimethoxybenzyl)-6-(4-ethoxyphenyl)pyrazine-2-carbohydrazide (1.05 mmol) in DCM (15.0 mL), NaBH(OAc)₃ (3.16 mmol) and formaldehyde (10.5 mmol) were added. The reaction mixture was kept at room temperature for 12 hours. After total consumption of the starting reagent (monitored by CCD), the mixture was diluted in water (20 mL) and extracted with DCM (5 mL×3). The organic phase was concentrated under reduced pressure and the residue was purified by chromatography column ([H₂O(NH₄HCO₃)-ACN]; B %: 37%-67%). 278 mg (62.5% yield) of the product of interest were obtained in the form of a white solid.

TABLE 11
COMPOUNDS OBTAINED ACCORDING TO SCHEDULE 1

| Compound | | ¹H-NMR/ |
|---|---|---|
| No | Structure | MS (m/z) [M + H]⁺ |
| 1 | 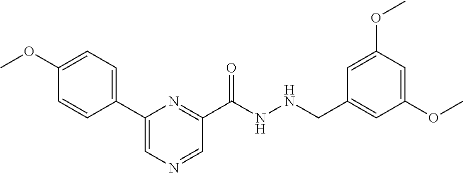 N′-(3,5-dimethoxybenzyl)-6-(4- methoxyphenyl)pyrazine-2- carbohydrazide | ¹H-NMR, 400 MHz, DMSO-d₆ δ = 10.43 (br s, 1H), 9.39 (s, 1H), 8.97 (s, 1H), 8.38-8.32 (m, 2H), 7.11- 7.06 (m, 2H), 6.61 (d, J = 2.2 Hz, 2H), 6.38 (t, J = 2.3 Hz, 1H), 5.58 (br s, 1H), 4.01 (br s, 2H), 3.85 (s, 3H), 3.72 (s, 6H). |
| 2 | 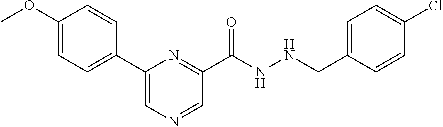 [SMILES: COc1ccc(-c2cncc(C(=O)NNCc3ccc(Cl)cc3)n2)cc1] N′-(4-chlorobenzyl)-6-(4- methoxyphenyl)pyrazine-2- carbohydrazide | ¹H-NMR, 400 MHz, DMSO-d₆ δ = 3.87 (s, 3 H) 4.08 (s, 2 H) 7.08- 7.15 (m, 2 h) 7.37-7.43 (m, 2 h) 7.44-7.50 (m, 2 h) 8.30-8.40 (m, 2 h) 8.97 (s, 1 h) 9.40 (s, 1 h) 10.46 (s, 1 h). [M + H]⁺: 369.0. |
| 3 | 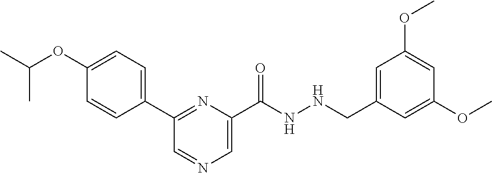 [SMILES: COc1cc(CNNC(=O)c2cncc(-c3ccc(OC(C)C)cc3)n2)cc(OC)c1] N′-(3,5-dimethoxybenzyl)-6-(4- isopropoxyphenyl)pyrazine-2- carbohydrazide | ¹H-NMR, 400 MHz, DMSO-d₆ δ = 10.46-10.36 (m, 1H), 9.42- 9.31 (m, 1H), 8.97 (s, 1H), 8.39- 8.24 (m, 2H), 7.13- 7.01 (m, 2H), 6.61 (d, J = 2.3 Hz, 2H), 6.42- 6.32 (m, 1H), 5.57 (q, J = 6.1 Hz, 1H), 4.84-4.68 (m, 1H), 4.01 (d, J = 5.9 Hz, 2H), 3.72 (s, 6H), 1.31 (d, J = 6.0 Hz, 6H). [M + H]⁺: 423.4. |
| 4 | 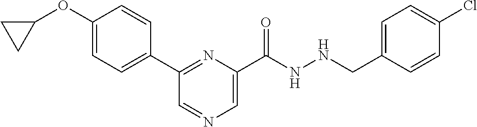 [SMILES: O=C(NNCc1ccc(Cl)cc1)c1cncc(-c2ccc(OC3CC3)cc2)n1] N′-(4-chlorobenzyl)-6-(4- cyclopropoxyphenyl)pyrazine-2- carbohydrazide | ¹H-NMR, 400 MHz, DMSO-d₆ δ = 10,42 (d, J = 6.4 Hz, 1H), 9.39 (s, 1H), 8.97 (s, 1H), 8.39-8.31 (m, 2H), 7.48- 7.44 (m, 2H), 7.42- 7.36 (m, 2H), 7.25-7.18 (m, 2H), 5.65 (q, J = 6.0 Hz, 1H), 4.07 (d, J = 6.0 Hz, 2H), 3.97 (tt, J = 2.9, 6.0 Hz, 1H), 0.90-0.80 (m, 2H), 0.74-0.68 (m, 2H). [M + H]⁺: 395.0. |
| 5 | 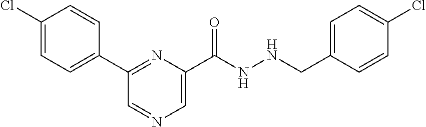 [SMILES: O=C(NNCc1ccc(Cl)cc1)c1cncc(-c2ccc(Cl)cc2)n1] N′-(4-chlorobenzyl)-6-(4- chlorophenyl)pyrazine-2- carbohydrazide | ¹H-NMR, 400 MHz, DMSO-d6 δ = 10.54 (d, J = 6.5 Hz, 1H), 9.48 (s, 1H), 9.07 (s, 1H), 8.45 (d, J = 8.7 Hz, 2H), 7.63 (d, J = 8.6 Hz, 2H), 7.48-7.42 (m, 2H), 7.41- 7.37 (m, 2H), 5.67 (d, J = 6.2 Hz, 1H), 4.07 (d, J = 6.0 Hz, 2H). [M + H]⁺: 373.0. |
| 6 | 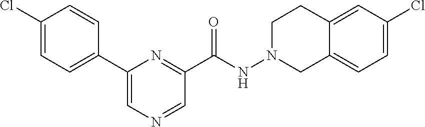 [SMILES: O=C(NN1CCc2cc(Cl)ccc2C1)c1cncc(-c2ccc(Cl)cc2)n1] N-(6-chloro-3,4- dihydroisoquinoline-2(1H)-yl)-6- (4-chlorophenyl)pyrazine-2- carboxamide | ¹H-NMR, 400 MHz, DMSO-d₆ δ = 10.24 (s, 1H), 9.51 (s, 1H), 9.11 (s, 1H), 8.45 (d, J = 8.6 Hz, 2H), 7.65 (d, J = 8.6 Hz, 2H), 7.33- 7.11 (m, 3H), 4.15 (s, 2H), 3.26 (br t, J = 6.0 Hz, 3H), 2.99 (br t, J = 5.7 Hz, 2H). [M + H]⁺: 399.0. |
| 7 | 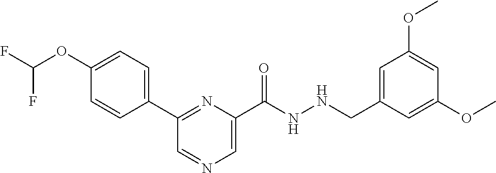 [SMILES: COc1cc(CNNC(=O)c2cncc(-c3ccc(OC(F)F)cc3)n2)cc(OC)c1] 6-(4-(difluoromethoxy) phenyl)-N′- (3,5-dimethoxybenzyl)pyrazine-2- carbohydrazide | ¹H-NMR, 400 MHz, DMSO-d₆ δ = 10.50 (s, 1H), 9.46 (s, 1H), 9.06 (s, 1H), 8.47 (d, J = 8.9 Hz, 2H), 7.64-7.16 (m, 3H), 6.60 (d, J = 2.0 Hz, 2H), 6.38 (t, J = 2.1 Hz, 1H), 5.77-5.44 (m, 1H), 4.01 (s, 2H), 3.71 (s, 6H). [M + H]⁺: 431.4. |
| 8 | 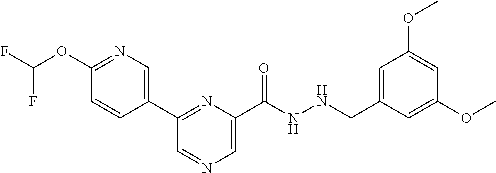 [SMILES: COc1cc(CNNC(=O)c2cncc(-c3ccc(OC(F)F)nc3)n2)cc(OC)c1] 6-(6-(difluoromethoxy)pyridine-3- yl)-N′-(3,5- dimethoxybenzyl)pyrazine-2- carbohydrazide | ¹H-NMR, 400 MHz, DMSO-d₆ δ = 10.60 (d, J = 6.5 Hz, 1H), 9.54 (s, 1H), 9.30 (d, J = 2.4 Hz, 1H), 9.11 (s, 1H), 8.95 (dd, J = 2.3, 8.7 Hz, 1H), 7.83 (t, J = 72.5 Hz, 1H), 7.30 (d, J = 8.8 Hz, 1H), 6.68- 6.31 (m, 3H), 5.61 (q, J = 6.0 Hz, 1H), 4.02 (d, J = 5.9 Hz, 2H), 3.72 (s, 6H). [M + H]⁺: 431.1. |
| 9 | 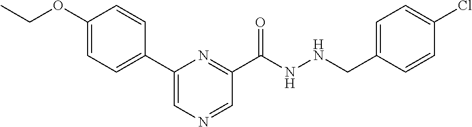 [SMILES: CCOc1ccc(-c2cncc(C(=O)NNCc3ccc(Cl)cc3)n2)cc1] N′-(4-chlorobenzyl)-6-(4- ethoxyphenyl)pyrazine-2- carbohydrazide | ¹H-NMR, 400 MHz, DMSO-d₆ δ = 10.43 (s, 1H), 9.39 (s, 1H), 8.96 (s, 1H), 8.42- 8.28 (m, 2H), 7.49- 7.43 (m, 2H), 7.42-7.36 (m, 2H), 7.12-7.04 (m, 2H), 4.14 (q, J = 7.0 Hz, 2H), 4.07 (s, 2H), 2.54- 2.53 (m, 1H), 1.38 (t, J = 7.0 Hz, 3H). [M + H]⁺: 383.0. |
| 10 | 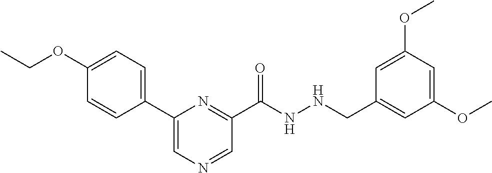 [SMILES: CCOc1ccc(-c2cncc(C(=O)NNCc3cc(OC)cc(OC)c3)n2)cc1] N′-(3,5-dimethoxybenzyl)-6-(4- ethoxyphenyl)pyrazine-2- carbohydrazide | ¹H-NMR, 400 MHz, DMSO-d₆ δ = 10.49 (s, 1H), 9.39 (s, 1H), 8.97 (s, 1H) , 8.33 (d, J = 8.8 Hz, 2H), 7.07 (d, J = 8.8 Hz, 2H), 6.61 (d, J = 2.2 Hz, 2H), 6.39 (t, J = 2.2 Hz, 1H), 4.13 (q, J = 7.1 Hz, 2H), 4.02 (s, 2H), 3.72 (s, 6H), 1.37 (t, J = 7.1 Hz, 3H). [M + H]⁺: 409.0. |
| 11 | 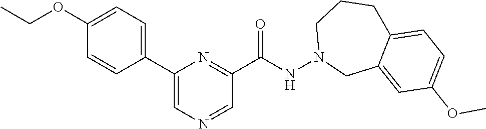 [SMILES: CCOc1ccc(-c2cncc(C(=O)NN3CCCc4ccc(OC)cc4C3)n2)cc1] 6-(4-ethoxyphenyl)-N-(8-methoxy- 1,3,4,5-tetrahydro-2H- benzo[c]azepine-2-yl)pyrazine-2- carboxamide | 1H-NMR, 400 MHz, CDCl₃ δ = 9.19 (s, 1H), 9.08 (s, 1H), 8.77 (s, 1H), 7.64- 7,. 57 (m, 2H), 7.21 (d, J = 8.3 Hz, 1H), 7.00- 6.96 (m, 2H), 6.91 (dd, J = 2.7, 8.2 Hz, 1H), 6.81 (d, J = 2.7 Hz, 1H), 4.35 (br s, 2H), 4.14 (q, J = 7.0 Hz, 2H), 3.72 (s, 3H), 3.58-3.50 (m, 2H), 3.01- 2.93 (m, 2H), 1.83- 1.77 (m, 2H), 1.49 (t, J = 7.0 Hz, 3H). [M + H]⁺: 419.2. |
| 12 | 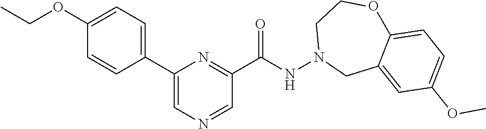 [SMILES: CCOc1ccc(-c2cncc(C(=O)NN3CCOc4ccc(OC)cc4C3)n2)cc1] 6-(4-ethoxyphenyl)-N-(7-methoxy- 2,3- dihydrobenzo[f][1,4]oxazepine- 4(5H)-yl)pyrazine-2-carboxamide | ¹H-NMR, 400 MHz, CDCl₃ δ = 9.12 (s, 1H), 9.03 (s, 1H), 8.91 (s, 1H), 7.63- 7.56 (m, 2H), 7.02 (d, J = 8.7 Hz, 1H), 6.93-6.82 (m, 3H), 6.74 (d, J = 3.1 Hz, 1H), 4.28 (s, 2H), 4.10- 4.01 (m, 4H), 3.68 (s, 3H), 3.53- 3.45 (m, 2H), 1.41 (t, J = 7.0 Hz, 3H). [M + H]⁺: 421.2. |
| 13 | 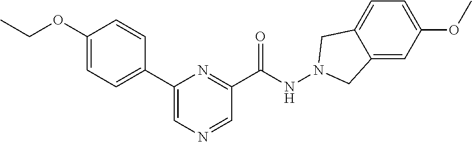 [SMILES: CCOc1ccc(-c2cncc(C(=O)NN3Cc4ccc(OC)cc4C3)n2)cc1] 6-(4-ethoxyphenyl)-N-(5- methoxyisoindoline-2-yl)pyrazine- 2-carboxamide | ¹H-NMR, 400 MHz, DMSO-d₆ δ = 1.37 (t, J = 6.8 Hz, 3 H) 3.76 (s, 3 H) 4.14 (q, J = 6.8 Hz, 2 H) 4.37 (s, 2 H), 4.20 (s, 2 H) 6.83- 6.89 (m, 2 H) 7.08-7.20 (m, 3 H) 4.37 (s, 2 h) 4.20 (s, 2 h) 8.37- 8.40 (m, 2 h) 9.01 (s, 1 H) 9.42 (s, 1 H) 10.35 (s, 1 H). [M + H]⁺: 391.2. |
| 14 | 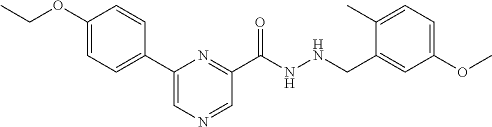 [SMILES: CCOc1ccc(-c2cncc(C(=O)NNCc3cc(OC)ccc3C)n2)cc1] 6-(4-ethoxyphenyl)-N′-(5-methoxy- 2-methylbenzyl)pyrazine-2- carbohydrazide | ¹H-NMR, 400 MHz, DMSO-d₆ δ = 1.37 (t, J = 6.94 Hz, 3 H) 2.30 (s, 3 H) 3.71 (s, 3 H) 3.91-4.20 (m, 4 H) 5.46 (s, 1 H) 6.74 (dd, J = 8.38, 2.75 Hz, 1 H) 6.86-7.20 (m, 4 H) 8.36 (d, J = 8.88 Hz, 2 H) 8.99 (s, 1 H) 9.39 (s, 1 H) 10.50 (d, J = 6.38 Hz, 1 H). [M + H]⁺: 393.4. |
| 15 | 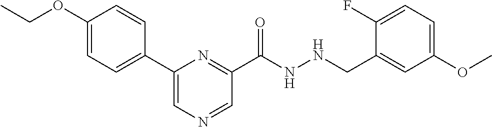 [SMILES: CCOc1ccc(-c2cncc(C(=O)NNCc3cc(OC)ccc3F)n2)cc1] 6-(4-ethoxyphenyl)-N′-(2-fluoro- 5-methoxybenzyl)pyrazine-2- carbohydrazide | ¹H-NMR, 400 MHz, DMSO-d₆ δ = 2.18-2.33 (m, 3 H) 3.53 (s, 3 H) 7.05-7.73 (m, 7 H) 8.08- 8.17 (m, 1 H) 8.98 (s, 1 H) 11.10- 11.19 (m, 1 H). [M + H]⁺: 397.4. |
| 16 | 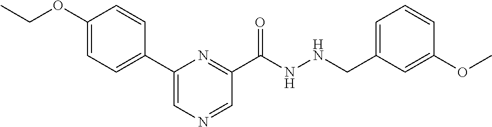 [SMILES: CCOc1ccc(-c2cncc(C(=O)NNCc3cccc(OC)c3)n2)cc1] 6-(4-ethoxyphenyl)-N′-(3- methoxybenzyl)pyrazine-2- carbohydrazide | ¹H-NMR, 400 MHz, DMSO-d₆ δ = 1.37 (t, J = 6.94 Hz, 3 H) 3.74 (s, 3 H) 3.96- 4.21 (m, 4 H) 5.55 (q, J = 6.21 Hz, 1 H) 7.35 (d, J = 12.13 Hz, 6 H) 8.33 (d, J = 8.88 Hz, 2 H) 8.84 (d, J = 4.50 Hz, 1 H) 9.38 (s, 1 H) 10.34-10.61 (m, 1 H). [M + H]⁺: 379.4. |
| 17 | 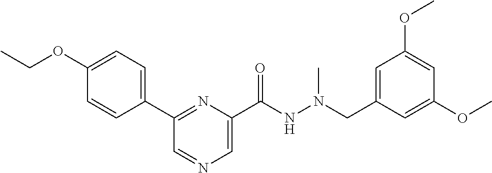 [SMILES: CCOc1ccc(-c2cncc(C(=O)NN(C)Cc3cc(OC)cc(OC)c3)n2)cc1] N′-(3,5-dimethoxybenzyl)-6-(4- ethoxyphenyl)-N′-methylpyrazine- 2-carbohydrazide | ¹H-NMR, 400 MHz, CDCl₃ δ = 9.23 (s, 1H), 9.10 (s, 1H), 8.68 (s, 1H), 7.98- 7.83 (m, 2H), 7.04 (d, J = 8.8 Hz, 2H), 6.58 (d, J = 2.3 Hz, 2H), 6.42 (t, J = 2.2 Hz, 1H), 4.22-4.02 (m, 4H), 3.76 (s, 6H), 2.82 (s, 3H), 1.48 (t, J = 7.0 Hz, 3H). [M + H]⁺: 423.5. |
| 18 | 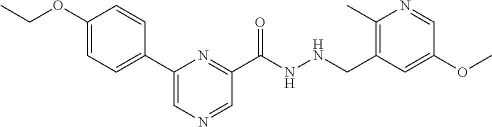 [SMILES: CCOc1ccc(-c2cncc(C(=O)NNCc3cc(OC)cnc3C)n2)cc1] 6-(4-ethoxyphenyl)-N′-((5- methoxy-2-methylpyridine-3- yl)methyl)pyrazine-2- carbohydrazide | 1H-NMR, 400 MHz, DMSO-d₆ δ = 1.30 (t, J = 6.94 Hz, 3 H) 2.50 (s, 3 H) 3.79 (s, 3 H) 4.04 (q, J = 6.84 Hz, 2 H) 4.20 (d, J = 1.38 Hz, 2 H) 6.94 (d, J = 8.88 Hz, 2 H) 7.22- 7.32 (m, 1 H) 7.47 (br d, J = 5.38 Hz, 1 H) 7.59 (d, J = 3.13 Hz, 1 H) 7.92 (d, J = 8.88 Hz, 2 H) 8.12 (d, J = 2.88 Hz, 1 H) 8.68 (s, 1 H) 10.85 (s, 1 H). [M + H]⁺: 394.1. |
| 19 | 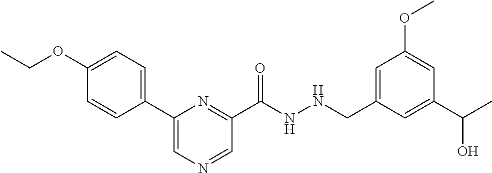 [SMILES: CCOc1ccc(-c2cncc(C(=O)NNCc3cc(OC)cc(C(C)O)c3)n2)cc1] 6-(4-ethoxyphenyl)-N′-(3-(1-hydroxyethyl)-5- methoxybenzyl)pyrazine-2-carbohydrazide | |
| 20 | 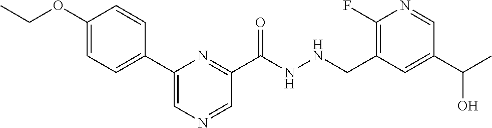 [SMILES: CCOc1ccc(-c2cncc(C(=O)NNCc3cc(C(C)O)cnc3F)n2)cc1] 6-(4-ethoxyphenyl)-N′-((2-fluoro-5-(1- hydroxyethyl)pyridine-3-yl)methyl)pyrazine-2- carbohydrazide | |
| 21 | 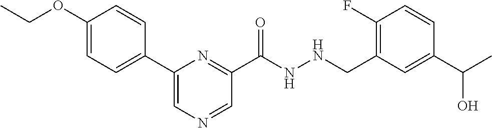 [SMILES: CCOc1ccc(-c2cncc(C(=O)NNCc3cc(C(C)O)ccc3F)n2)cc1] 6-(4-ethoxyphenyl)-N′-(2-fluoro-5-(1- hydroxyethyl)benzyl)pyrazine-2-carbohydrazide | |
| 22 | 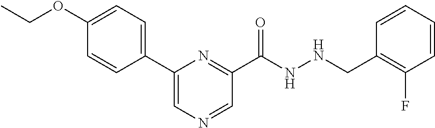 [SMILES: CCOc1ccc(-c2cncc(C(=O)NNCc3ccccc3F)n2)cc1] 6-(4-ethoxyphenyl)-N′-(2- fluorobenzyl)pyrazine-2- carbohydrazide | ¹H-NMR, 400 MHz, DMSO-d₆ δ = 1.37 (t, J = 6.94 Hz, 3 H) 4.05- 4.17 (m, 4 H) 5.49-5.66 (m, 1 H) 7.02-7.21 (m, 4 H) 7.27-7.38 (m, 1 H) 7.47- 7.64 (m, 1 H) 8.35 (d, J = 8.75 Hz, 2 H) 8.96 (s, 1 H) 9.38 (s, 1 H) 10.51 (d, J = 6.50 Hz, 1 H). [M + H]⁺: 367.4. |
| 23 | 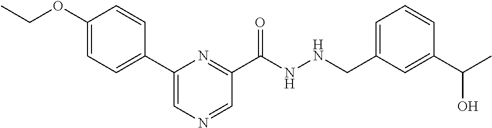 [SMILES: CCOc1ccc(-c2cncc(C(=O)NNCc3cccc(C(C)O)c3)n2)cc1] 6-(4-ethoxyphenyl)-N′-(3-(1- hydroxyethyl)benzyl)pyrazine-2-carbohydrazide. | |
| 24 | 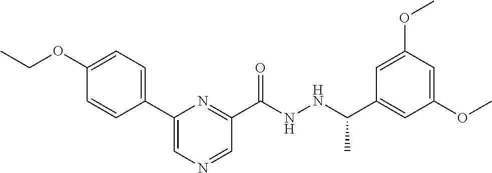 [SMILES: CCOc1ccc(-c2cncc(C(=O)NN[C@@H](C)c3cc(OC)cc(OC)c3)n2)cc1] (S*)-N′-(1-(3,5- dimethoxyphenyl)ethyl)-6-(4- ethoxyphenyl)pyrazine-2- carbohydrazide1 | ¹H-NMR, 400 MHz, CDCl₃ δ = 9.19 (s, 1H), 9.11 (s, 1H), 9.01 (s, 1H), 7.94- 7.78 (m, 2H), 7.08- 6.94 (m, 2H), 6.67-6.58 (m, 2H), 6.42 (t, J = 2.3 Hz, 1H), 5.33- 4.77 (m, 1H), 4.23 (q, J = 6.6 Hz, 1H), 4.12 (q, J = 7.0 Hz, 2H), 3.80 (s, 6H), 1.52 1.43 (m, 6H). [M + H]⁺: 423.5. |
| 25 | 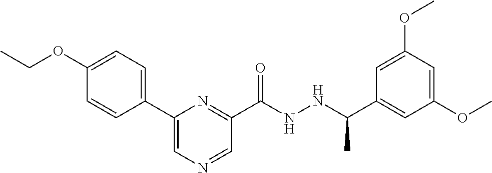 [SMILES: CCOc1ccc(-c2cncc(C(=O)NN[C@H](C)c3cc(OC)cc(OC)c3)n2)cc1] (R*)-N′-(1-(3,5- dimethoxyphenyl)ethyl)-6-(4- ethoxyphenyl)pyrazine-2- carbohydrazide1 | ¹H-NMR, 400 MHz, CDCl₃ δ = 9.21-9.07 (m, 2H), 7.91- 7.79 (m, 2H), 7.01 (d, J = 8.9 Hz, 2H), 6.62 (d, J = 2.3 Hz, 2H), 6.42 (t, J = 2.3 Hz, 1H), 4.24 (q, J = 6.5 Hz, 1H), 4.12 (q, J = 6.9 Hz, 2H), 3.86-3.68 (m, 6H), 1.52- 1.42 (m, 6H). [M + H]⁺: 423.5. |
| 26 | 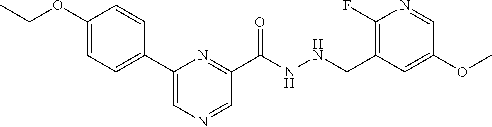 [SMILES: CCOc1ccc(-c2cncc(C(=O)NNCc3cc(OC)cnc3F)n2)cc1] 6-(4-ethoxyphenyl)-N′-((2-fluoro- 5-methoxypyridine-3- yl)methyl)pyrazine-2- carbohydrazide | ¹H-NMR, 400 MHz, DMSO-d₆ δ = 1.37 (t, J = 6.94 Hz, 3 H) 3.80 (s, 3 H) 4.06- 4.17 (m, 4 H) 5.83 (q, J = 5.63 Hz, 1 H) 7.07 (d, J = 8.76 Hz, 2 H) 7.74- 7.78 (m, 2 H) 8.34 (d, J = 8.76 Hz, 2 H) 8.95 (s, 1 H) 9.38 (s, 1 H) 10.48 (d, J = 6.00 Hz, 1 H). [M + H]⁺: 398.1. |
| 27 | 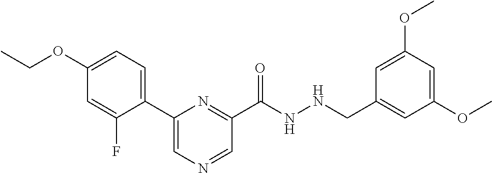 [SMILES: CCOc1ccc(-c2cncc(C(=O)NNCc3cc(OC)cc(OC)c3)n2)c(F)c1] N′-(3,5-dimethoxybenzyl)-6-(4- ethoxy-2-fluorophenyl)pyrazine-2- carbohydrazide | ¹H-NMR, 400 MHz, DMSO-d₆ δ = 10.38 (d, J = 6,4 Hz, 1H), 9.15 (d, J = 2.0 Hz, 1H), 9.02 (s, 1H), 8.42-8.22 (m, 1H), 7.09-6.90 (m, 2H), 6.60 (d, J = 2.1 Hz, 2H), 6.38 (s, 1H), 5.58 (q, J = 6.0 Hz, 1H), 4.15 (q, J = 6.9 Hz, 2H), 4.03- 3.97 (m, 2H), 3.71 (s, 6H), 1.37 (t, J = 6.9 Hz, 3H). [M + H]⁺: 427. 4. |
| 28 | 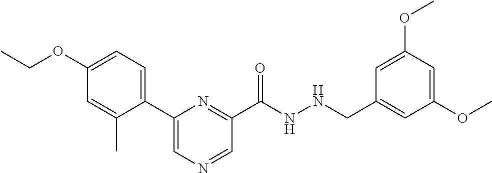 [SMILES: CCOc1ccc(-c2cncc(C(=O)NNCc3cc(OC)cc(OC)c3)n2)c(C)c1] N′-(3,5-dimethoxybenzyl)-6-(4- ethoxy-2-methylphenyl)pyrazine-2- carbohydrazide | ¹H-NMR, 400 MHz, DMSO-d₆ δ = 10,07 (d, J = 6.4 Hz, 1H), 9.08- 8.89 (m, 2H), 7.54 (d, J = 8.4 Hz, 1H), 6.99- 6.83 (m, 2H), 6.57 (d, J = 2.1 Hz, 2H), 6.36 (t, J = 2.1 Hz, 1H), 5.60 (q, J = 5.8 Hz, 1H), 4.09 (q, J = 6.9 Hz, 2H), 3.96 (d, J = 5.6 Hz, 2H), 3.70 (s, 6H), 2.34 (s, 3H), 1.35 (t, J = 6.9 Hz, 3H). [M + H]⁺: 423.4. |
| 29 | 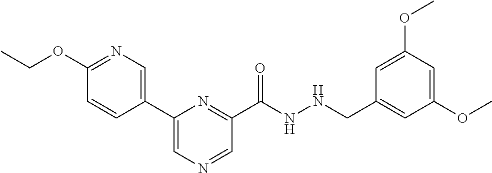 [SMILES: CCOc1ccc(-c2cncc(C(=O)NNCc3cc(OC)cc(OC)c3)n2)cn1] N′-(3,5-dimethoxybenzyl)-6-(6- ethoxypyridine-3-yl)pyrazine-2- carbohydrazide | ¹H-NMR, 400 MHz, DMSO-d₆ δ = 10.52 (d, J = 6.4 Hz, 1H), 9.45 (s, 1H), 9.19 (d, J = 2.1 Hz, 1H), 9.03 (s, 1H), 8.72 (dd, J = 2.5, 8.8 Hz, 1H), 6.96 (d, J = 8.8 Hz, 1H), 6.60 (d, J = 2.3 Hz, 2H), 6.37 (t, J = 2.3 Hz, 1H), 5.58 (q, J = 6.1 Hz, 1H), 4.40 (q, J = 7.0 Hz, 2H), 4.10-3.94 (m, 2H), 3.71 (s, 6H), 1.36 (t, J = 7.1 Hz, 3H). [M + H]⁺: 409.1. |
| 30 | 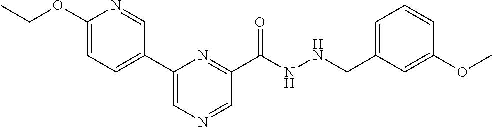 [SMILES: CCOc1ccc(-c2cncc(C(=O)NNCc3cccc(OC)c3)n2)cn1] 6-(6-ethoxypyridine-3-yl)-N′-(3- methoxybenzyl)pyrazine-2- carbohydrazide | ¹H-NMR, 400 MHz, DMSO-d₆ δ = 1,36 (t, J = 7.00 Hz, 3 H) 3.73 (s, 3 H) 4.04 (br d, J = 5.75 Hz, 2 H) 4.40 (q, J = 7.05 Hz, 2 H) 5.57 (q, J = 6.00 Hz, 1 H) 6.82 (dd, J = 8.19, 2.19 Hz, 1 H) 6.93-7.04 (m, 3 H) 7.21-7.26 (m, 1 H) 8.72 (dd, J = 8.76, 2.50 Hz, 1 H) 9.03 (s, 1 H) 9.20 (d, J = 2.25 Hz, 1 H) 9.45 (s, 1 H) 10.55 (br d, J = 6.50 Hz, 1 H). [M + H]⁺: 380.4. |
| 31 | 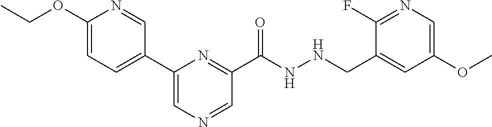 [SMILES: CCOc1ccc(-c2cncc(C(=O)NNCc3cc(OC)cnc3F)n2)cn1] 6-(6-ethoxypyridine-3-yl)-N′-((2- fluoro-5-methoxypyridine-3- yl)methyl)pyrazine-2- carbohydrazide | ¹H-NMR, 400 MHz, DMSO-d₆ δ = 1.33-1.38 (m, 3 H) 3,3 80 (d, J = 1.50 Hz, 3 H) 4.09 (br d, J = 4.75 Hz, 2 H) 4.37- 4.43 (m, 2 H) 5.79-5.85 (m, 1 H) 6.93-6.99 (m, 1 H) 7.71-7.79 (m, 2 H) 8.68- 8.74 (m, 1 H) 9.01 (d, J = 1.25 Hz, 1 H) 9.15-9.22 (m, 1 H) 9.42-9.48 (m, 1 H) 10.53 (br d, J = 6.38 Hz, 1 H). [M + H]⁺: 399.4. |
| 32 | 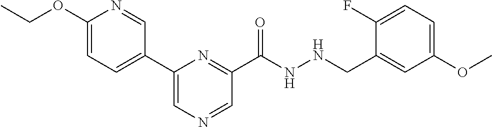 [SMILES: CCOc1ccc(-c2cncc(C(=O)NNCc3cc(OC)ccc3F)n2)cn1] 6-(6-ethoxypyridine-3-yl)-N′-(2- fluoro-5-methoxybenzyl)pyrazine- 2-carbohydrazide | ¹H-NMR, 400 MHz, DMSO-d₆ δ = 1.33-1.40 (m, 3 H) 3.71 (br s, 3 h) 4.09 (br s, 2 h) 4.37- 4.44 (m, 2 h) 5.65 (br d, J = 1.38 Hz, 1 h) 6.79- 6.86 (m, 1 h) 6.93- 6.99 (m, 1 h) 7.04-7.16 (m, 2 h) 8.68-8.75 (m, 1 h) 9.00-9.05 (m, 1 H) 9.19 (br s, 1 H) 9.42- 9.48 (m, 1 H) 10.52-10.59 (m, 1 H). [M + H]⁺: 398.4. |
| 33 | 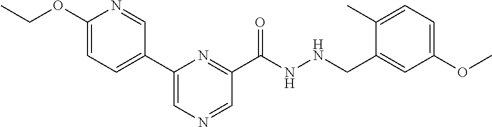 [SMILES: CCOc1ccc(-c2cncc(C(=O)NNCc3cc(OC)ccc3C)n2)cn1] 6-(6-ethoxypyridine-3-yl)-N′-(5- methoxy-2-methylbenzyl)pyrazine- 2-carbohydrazide | ¹H-NMR, 400 MHz, DMSO-d₆ δ = 1.36 (t, J = 7.07 Hz, 3 H) 2.29 (s, 3 H) 3.71 (s, 3 H) 4.01 (d, J = 6.25 Hz, 2 H) 4.41 (q, J = 7.09 Hz, 2 H) 5.41 (q, J = 6.38 Hz, 1 H) 6.74 (dd, J = 8.25, 2.75 Hz, 1 H) 6.94- 7.03 (m, 2 H) 7.07 (d, J = 8.38 Hz, 1 H) 8.73 (dd, J = 8.76, 2.50 Hz, 1 H) 9.05 (s, 1 H) 9.21 (d, J = 2.13 Hz, 1 H) 9.46 (s, 1 H) 10.58 (d, J = 6.50 Hz, 1 H). [M + H]⁺: 394.4. |
| 34 | 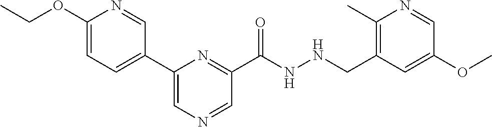 [SMILES: CCOc1ccc(-c2cncc(C(=O)NNCc3cc(OC)cnc3C)n2)cn1] 6-(6-ethoxypyridine-3-yl)-N′-((5- methoxy-2-methylpyridine-3- yl)methyl)pyrazine-2- carbohydrazide | ¹H-NMR, 400 MHz, DMSO-d₆ δ = 1.31-1.37 (m, 3 H) 2.56 (s, 3 H) 3.85 (s, 3 H) 4.30 (d, J = 1.50 Hz, 2 H) 4.37 (q, J = 7.05 Hz, 2 H) 6.89 (d, J = 8.75 Hz, 1 H) 7.34 (d, J = 6.13 Hz, 1 H) 7.58-7.70 (m, 2 H) 8.18 (d, J = 3.00 Hz, 1 H) 8.49 (dd, J = 8.76, 2.50 Hz, 1 H) 8.66- 8.75 (m, 2 H) 10.93 (s, 1 H). [M + H]⁺: 395.4. |
| 35 | 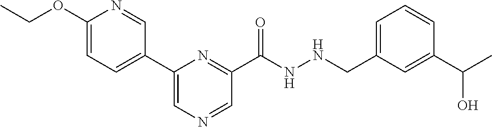 [SMILES: CCOc1ccc(-c2cncc(C(=O)NNCc3cccc(C(C)O)c3)n2)cn1] 6-(6-ethoxypyridine-3-yl)-N′-(3-(1- hydroxyethyl)benzyl)pyrazine-2-carbohydrazide | |
| 36 | 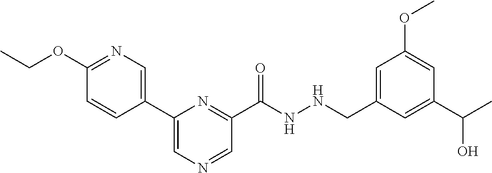 [SMILES: CCOc1ccc(-c2cncc(C(=O)NNCc3cc(OC)cc(C(C)O)c3)n2)cn1] 6-(6-ethoxypyridine-3-yl)-N′-(3-(1-hydroxyethyl)-5- methoxybenzyl)pyrazine-2-carbohydrazide | |
| 37 | 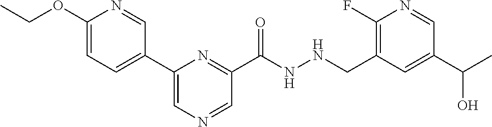 [SMILES: CCOc1ccc(-c2cncc(C(=O)NNCc3cc(C(C)O)cnc3F)n2)cn1] 6-(6-ethoxypyridine-3-yl)-N′-((2-fluoro-5-(1- hydroxyethyl)pyridine-3-yl)methyl)pyrazine-2- carbohydrazide | |
| 38 | 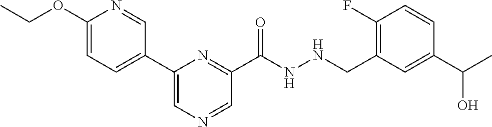 [SMILES: CCOc1ccc(-c2cncc(C(=O)NNCc3cc(C(C)O)ccc3F)n2)cn1] 6-(6-ethoxypyridine-3-yl)-N′-(2-fluoro-5-(1- hydroxyethyl)benzyl)pyrazine-2-carbohydrazide | |
| 39 | 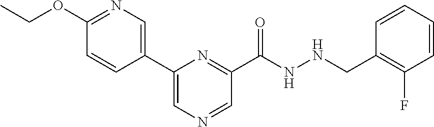 [SMILES: CCOc1ccc(-c2cncc(C(=O)NNCc3ccccc3F)n2)cn1] 6-(6-ethoxypyridine-3-yl)-N′-(2- fluorobenzyl)pyrazine-2- carbohydrazide | ¹H-NMR, 400 MHz, DMSO-d₆ δ = 1.37 (q, J = 7.09 Hz, 3 H) 4.12 (br s, 2 h) 4.37- 4.46 (m, 2 h) 5.57- 5.67 (m, 1 h) 6.93-7.01 (m, 1 h) 7.12-7.22 (m, 2 h) 7.27-7.37 (m, 1 h) 7.52- 7.60 (m, 1 h) 8.69- 8.77 (m, 1 h) 8.99-9.05 (m, 1 h) 9.21 (br d, J = 4.88 Hz, 1 H) 9.46 (br d, J = 7.88 Hz, 1 H) 10.59 (br t, J = 6.82 Hz, 1 H). [M + H]⁺: 368.3. |
| 40 | 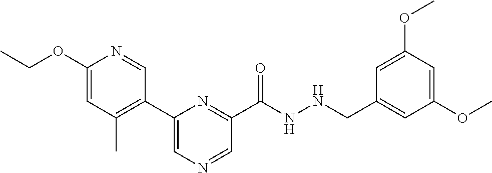 [SMILES: CCOc1cc(C)c(-c2cncc(C(=O)NNCc3cc(OC)cc(OC)c3)n2)cn1] N′-(3,5-dimethoxybenzyl)-6-(6- ethoxy-4-methylpyridine-3- yl)pyrazine-2-carbohydrazide | ¹H-NMR, 400 MHz, DMSO-d₆ δ = 9.33 (s, 1H), 8.89 (s, 1H), 8.22 (s, 1H), 6.71 (s, 1H), 6.57 (d, J = 1.8 Hz, 2H), 6.44- 6.37 (m, 1H), 4.44 (q, J = 7.0 Hz, 2H), 4.08 (s, 2H), 3.79 (s, 6H), 2.32 (s, 3H), 1.44 (t, J = 7.1 Hz, 3H) . [M + H]⁺: 423.4. |
| 41 | 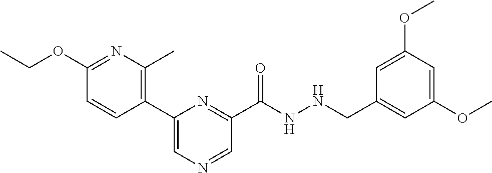 [SMILES: CCOc1ccc(-c2cncc(C(=O)NNCc3cc(OC)cc(OC)c3)n2)c(C)n1] N′-(3,5-dimethoxybenzyl)-6-(6- ethoxy-2-methylpyridine-3- yl)pyrazine-2-carbohydrazide | ¹H-NMR, 400 MHz, DMSO-d₆ δ = 10.15 (d, J = 6.5 Hz, 1H), 9.04 (d, J = 9.5 Hz, 2H), 8.00 (d, J = 8.5 Hz, 1H), 6.86- 6.74 (m, 1H), 6.57 (d, J = 2.3 Hz, 2H), 6.36 (t, J = 2.3 Hz, 1H), 5.60 (q, J = 5.9 Hz, 1H), 4.37 (q, J = 7.0 Hz, 2H), 3.97 (d, J = 5.8 Hz, 2H), 3.70 (s, 6H), 2.49 (br s, 3H), 1.34 (t, J = 7.1 Hz, 3H). [M + H]⁺: 423.4. |
| 42 | 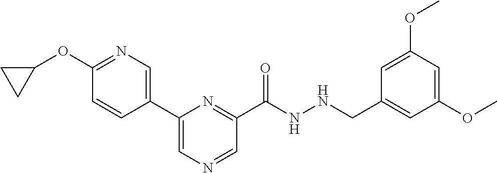 [SMILES: COc1cc(CNNC(=O)c2cncc(-c3ccc(OC4CC4)nc3)n2)cc(OC)c1] 6-(6-cyclopropoxypyridine-3-yl)- N′-(3,5-dimethoxybenzyl)pyrazine- 2-carbohydrazide | ¹H-NMR, 400 MHz, DMSO-d₆ δ = 10.52 (br d, J = 6.1 Hz, 1H), 9.46 (s, 1H), 9.23 (s, 1H), 9.04 (s, 1H), 8.73 (br dd, J = 1.7, 8.6 Hz, 1H), 7.02 (br d, J = 8.6 Hz, 1H), 6.60 (s, 2H), 6.37 (br s, 1H), 5.66- 5.49 (m, 1H), 4.31 (br d, J = 2.9 Hz, 1H), 4.01 (br d, J = 5.5 Hz, 2H), 3.71 (s, 6H), 0.89- 0.62 (m, 4H) . [M + H]⁺: 421.4. |
| 43 | 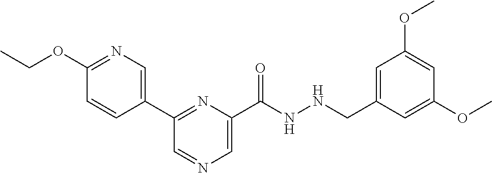 N′-(3,5-dimethoxybenzyl)-6-(6- isopropoxypyridine-3-yl)pyrazine- 2-carbohydrazide | ¹H-NMR, 400 MHz, DMSO-d₆ δ = 10.52 (d, J = 6.5 Hz, 1H), 9.44 (s, 1H), 9.19 (d, J = 2.4 Hz, 1H), 9.02 (s, 1H), 8.70 (dd, J = 2.4, 8.8 Hz, 1H), 6.90 (d, J = 8.6 Hz, 1H), 6.60 (d, J = 2.1 Hz, 2H), 6.37 (t, J = 2.1 Hz, 1H), 5.58 (q, J = 6.0 Hz, 1H), 5.35 (q, J = 6.2 Hz, 1H), 4.01 (d, J = 6.0 Hz, 2H), 3.71 (s, 6H), 1.34 (d, J = 6.3 Hz, 6H). [M + H]⁺: 423.4. |
| 44 | 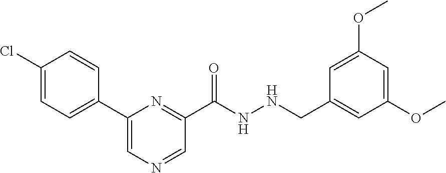 [SMILES: COc1cc(CNNC(=O)c2cncc(-c3ccc(Cl)cc3)n2)cc(OC)c1] 4-(4-chlorophenyl)-N′-(3,5- dimethoxybenzyl)pyrimidine-2- carbohydrazide | ¹H-NMR, 400 MHz, DMSO-d₆ δ 10.44 (s, 1H), 8.99 (d, J = 5.4 Hz, 1H), 8.43- 8.36 (m, 2H), 8.24 (d, J = 5.4 Hz, 1H), 7.69-7.62 (m, 2H), 6.61 (d, J = 2.3 Hz, 2H), 6.38 (t, J = 2.3 Hz, 1H), 4.01 (s, 2H), 3.72 (s, 6H). |
| 45 | 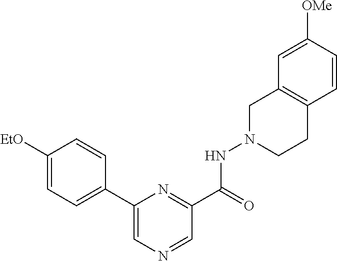 [SMILES: CCOc1ccc(-c2cncc(C(=O)NN3CCc4ccc(OC)cc4C3)n2)cc1] 6-(4-ethoxyphenyl)-N-(7-methoxy- 3,4-dihydroisoquinoline-2(1H)- yl)pyrazine-2-carboxamide | ¹H-NMR, 400 MHz, DMSO-d₆ δ = 1.36 (t, J = 6.8 Hz, 3 H) 2.87- 2.90 (m, 2 H), 3.21-3.24 (m, 2 H) 3.70 (s, 3 H) 4.09-4.15 (m, 4 H), 6.67-6.76 (m, 2 H), 7.01- 7.09 (m, 3 H) 8.32- 8.34 (m, 2 H), 8.99 (s, 1 h) 9.39 (s, 1 h) 10.06 (s, 1 H). [M + H]⁺: 405.2. |
| 46 | 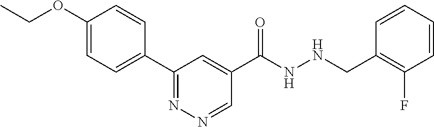 [SMILES: CCOc1ccc(-c2cc(C(=O)NNCc3ccccc3F)cnn2)cc1] 6-(4-ethoxyphenyl)-N′-(2- fluorobenzyl)pyridazine-4- carbohydrazide | ¹H NMR, 400 MHz, DMSO-d₆ δ: 10,55 (br s, 1H), 9,35 (d, J = 2,0 Hz, 1H), 8,36 (d, J = 2,0 Hz, 1H), 8,16-8,12 (m, 2H), 7,53-7,49 (m, 1H), 7,36-7,31 (m, 1H), 7,20-7,17 (m, 4H), 5,76 (br s, 1H), 4,14 (q, J = 6,8 Hz, 2H), 4,11-4,08 (m, 2H), 1,38 (t, J = 6,8 Hz, 3H). [M + H]⁺: 367,21. |
| 47 | 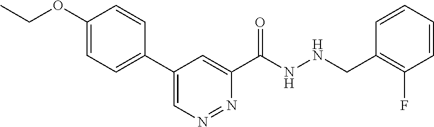 [SMILES: CCOc1ccc(-c2cnnc(C(=O)NNCc3ccccc3F)c2)cc1] 5-(4-ethoxyphenyl)-N′-(2- fluorobenzyl)pyridazine-3- carbohydrazide | ¹H NMR, 400 MHz, DMSO-d₆ δ: 11,71 (s, 1H), 10,70 (d, J = 6,0 Hz, 1H), 9,76 (d, J = 2,4 Hz, 1H), 8,27 (d, J = 2,4 Hz, 1H), 7,99-7,95 (m, 2H), 7,59-7,,55 (m, 1H), 7,32-7,28 (m, 1H), 7,18-7,09 (m, 4H), 5,69 (dd, J = 6,0 Hz, 12,0 Hz, 1H), 4,15 (m, 4H), 1,36 (t, J = 6,8 Hz, 3H). [M + H]⁺: 367,24. |
| 48 | 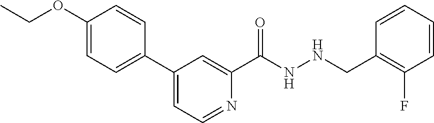 [SMILES: CCOc1ccc(-c2ccnc(C(=O)NNCc3ccccc3F)c2)cc1] 4-(4-ethoxyphenyl)-N′-(2- fluorobenzyl)picolinohydrazide | ¹H NMR, 400 MHz, CDCl₃ δ 8.51 (d, J = 5.14 Hz, 1H), 8.40 (d, J = 1.35 Hz, 1H), 7.68 (d, J = 8.80 Hz, 2H), 7.63 (dd, J = 5.07, 1.77 Hz, 1H), 7.50- 7.44 (m, 1H), 7.34-7.28 (m, 1H), 7.18-7.12 (m, 1H), 7.08 (t, J = 9.17 Hz, 1H), 7.02 (d, J = 8.80 Hz, 2H), 4.22 (s, 2H), 4.15-4.07 (m, 2H), 1.46 (t, J = 6.97 Hz, 3H). [M + H]⁺: 366.1. |
| 49 | 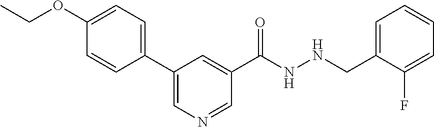 [SMILES: CCOc1ccc(-c2cncc(C(=O)NNCc3ccccc3F)c2)cc1] 5-(4-ethoxyphenyl)-N′-(2- fluorobenzyl)nicotinohydrazide | ¹H NMR, 400 MHz, DMSO-d₆ δ 10.29 (br d, J = 6.13 Hz, 1H), 8.97 (d, J = 2.13 Hz, 1H), 8.84 (d, J = 1.88 Hz, 1H), 8.32 (t, J = 2.06 Hz, 1H), 7.75-7.69 (m, 2H), 7.50 (td, J = 7.60, 1.81 Hz, 1H), 7.36-7.29 (m, 1H), 7.21- 7.12 (m, 2H), 7.07 (d, J = 8.76 Hz, 2H), 5.64 (br d, J = 5.63 Hz, 1H), 4.13-4.03 (m, 4H), 1.35 (t, J = 6.94 Hz, 3H). [M + H]⁺: 366.1. |
| 50 | 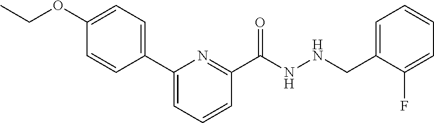 [SMILES: CCOc1ccc(-c2cccc(C(=O)NNCc3ccccc3F)n2)cc1] 6-(4-ethoxyphenyl)-N′-(2- fluorobenzyl)picolinohydrazide | ¹H NMR, 400 MHz, DMSO-d₆ δ 10.24 (d, J = 6.75 Hz, 1H), 8.24 (d, J = 8.76 Hz, 2H), 8.10-8.04 (m, 1H), 7.98 (t, J = 7.82 Hz, 1H), 7.84 (d, J = 7.38 Hz, 1H), 7.55 (td, J = 7.63, 1.63 Hz, 1H), 7.37-7.29 (m, 1H), 7.21- 7.13 (m, 2H), 7.02 (d, J = 8.88 Hz, 2H), 5.54 (q, J = 6.21 Hz, 1H), 4.15- 4.07 (m, 4H), 1.36 (t, J = 6.94 Hz, 3H). [M + H]⁺: 366.1. |
| 51 | 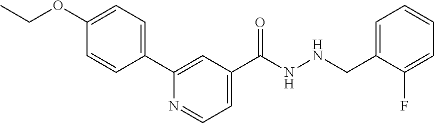 [SMILES: CCOc1ccc(-c2cc(C(=O)NNCc3ccccc3F)ccn2)cc1] 2-(4-ethoxyphenyl)-N′-(2- fluorobenzyl)isonicotinohydrazide | ¹H NMR, 400 MHz, DMSO-d₆ δ 10.40 (d, J = 6.25 Hz, 1H), 8.72 (dd, J = 5.00, 0.63 Hz, 1H), 8.16 (s, 1H), 8.10- 8.03 (m, 2H), 7.58 (dd, J = 5.00, 1.50 Hz, 1H), 7.50 (td, J = 7.60, 1.81 Hz, 1H), 7.37- 7.29 (m, 1H), 7.22-7.14 (m, 2H), 7.09-7.03 (m, 2H), 5.67 (q, J = 5.50 Hz, 1H), 4.15-4.05 (m, 4H), 1.36 (t, J = 7.00 Hz, 3H) . [M + H]⁺: 366.1. |
| 52 | 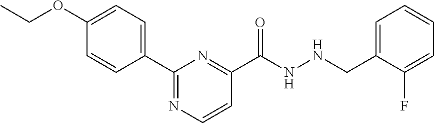 [SMILES: CCOc1ccc(-c2nccc(C(=O)NNCc3ccccc3F)n2)cc1] 2-(4-ethoxyphenyl)-N′-(2- fluorobenzyl)pyrimidine-4- carbohydrazide | ¹H NMR, 400 MHz, DMSO-d₆ δ 10.65 (br d, J = 6,. 38 Hz, 1H), 9.03 (d, J = 4.88 Hz, 1H), 8.59- 8.53 (m, 2H), 7.75 (d, J = 4.88 Hz, 1H), 7.54 (td, J = 7.66, 1.81 Hz, 1H), 7.32 (tdd, J = 7.69, 7.69, 5.63, 1.75 Hz, 1H), 7.21-7.13 (m, 2H), 7.08- 7.02 (m, 2H), 5.63 (q, J = 6.25 Hz, 1H), 4.17-4.09 (m, 4H), 1.37 (t, J = 7.00 Hz, 3H). [M + H]⁺: 367.1. |
| 53 | 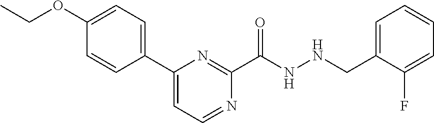 [SMILES: CCOc1ccc(-c2ccnc(C(=O)NNCc3ccccc3F)n2)cc1] 4-(4-ethoxyphenyl)-N′-(2- fluorobenzyl)pyrimidine-2- carbohydrazide | ¹H NMR, 400 MHz, DMSO-d₆ δ 10.39 (d, J = 6, 75 Hz, 1H), 8.86 (d, J = 5.38 Hz, 1H), 8.32 (d, J = 8.88 Hz, 2H), 8.11 (d, J = 5.50 Hz, 1H), 7.55 (td, J = 7.63, 1.75 Hz, 1H), 7.32 (tdd, J = 7.71, 7.71, 5.60, 1.75 Hz, 1H), 7.21-7.13 (m, 2H) , 7.09 (d, J = 9.01 Hz, 2H), 5.61 (q, J = 6.09 Hz, 1H), 4.18- 4.07 (m, 4H), 1.37 (s, 3H). [M + H]⁺: 367.1. |

¹ Relative Stereochemistry

### Example 3. Tests In Vitro

Biological assays were performed on Chinese hamster ovary (CHO) cells that express human sodium channels Nav 1.8 or Nav 1.7 in a stable manner.

The experimental protocol voltage-clamp using the whole cell configuration was established on the automated ScreenPatch© 384P platform (SP384PE, Nanion Technologies, Livingston, NJ) and the results were recorded with a Nanion 384-well Patch Clamp chip (NPC) (Nanion Technologies, Livingston, NJ).

Formula I compounds were diluted in eight concentrations in the extracellular solution composed of saline saline solution, buffered with HEPES (mM): NaCl, 137; KCl, 4; CaCl₂, 3.8; MgCl₂, 1; HEPES, 10; Glucose, 10; pH 7.4. The extracellular solution is composed of (mM) CsCl, 50; CsF, 90; MgCl₂, 5; EGTA, 5; HEPES, 10; pH 7.2. The duration of exposure of each compound with cells expressing Nav 1.7 or Nav 1.8 was at least five minutes, and the assays were performed at room temperature.

The measurements of the sodium currents of Nav 1.8 and Nav 1.7 were obtained using the voltage protocol described below.

A holding voltage of −100 mV was established followed by an inactivation voltage step at −40 mV for 8 seconds, followed by a step of −100 mV for 20 ms, followed by a 20 ms step for 10 mV for Nav 1.8 or 0 mV for Nav 1.7 (TP1A) before returning to the holding voltage of −100 mV.

The protocol was repeated at a frequency of 0.05 Hz and the amplitude of the current was quantified throughout the recording of the TP1A phase. The variation in the amplitude of the peak current was evaluated according to the formula described below, after exposing the cells expressing the channels to each concentration of the different compounds:

$$\% Block = ( 1 - ( {I}_{TP 1 A , molecule} / {I}_{TP 1 A , basal} ) \times 100 \% ,$$

wherein I_{TP1A,basal} and I_{TP1A,molecule} represent the peaks of sodium current input into TP1A before exposure to the compound and in the presence of the compound, respectively.

The decrease in the amplitude of the peak current, after the exposure of the cells to the compounds, was used to calculate the percentage of relative blockage of the channels in relation to the positive control according to the formula below:

$$\% {Block}^{′} = 100 \% - ( ( \% Block - \% CP ) * ( 100 \% / ( \% V - \% CP ) ) ,$$

wherein % V and % CP represent the means of the values of current inhibition with a vehicle (DMSO) and positive controls, respectively. The sodium currents of the positive control were considered as 100%.

$$\% {Block}^{′} = ( 100 \% / [ 1 + {( [ Test ] / {CI}_{50} )}^{N} ] ,$$

wherein [Test] represents the concentration of the molecule evaluated, CI₅₀ is the concentration of the compound that generates half of the maximum inhibition, N is the Hill coefficient, and % Block′ is the percentage of sodium channel current (Nav 1.8 and Nav 1.7) blocked at each concentration of the molecule evaluated. Data were obtained by nonlinear regression (nonlinear least squares) with XLfit for Excel (Microsoft, Redmond, WA).

The compounds were tested in at least one assay to obtain the value of CI₅₀. For compounds that were tested in two or more assays, the results are described as the means of the CI₅₀ values.

Table 5 shows the efficacy in vitro of selected compounds against Nav1.8 and Nav1.7. C₅₀ values less than 500 nM are represented with the legend (+++); CI₅₀ values between 500 nM and 1000 nM are represented with the legend (++), CI₅₀ values greater than 1000 nM are demonstrated with the symbology (+)

TABLE 5
CI₅₀ VALUES OF THE COMPOUNDS OF THIS INVENTION
IN NAV 1.8 AND NAV 1.7 CHANNELS.

| | Nav1.8 IC₅₀ | Nav1.7 IC₅₀ |
|---|---|---|
| Compound No | (nM) | (nM) |
| 1 | ++ | + |
| 2 | + | + |
| 3 | +++ | + |
| 4 | + | + |
| 5 | + | + |
| 6 | + | + |
| 7 | ++ | + |
| 8 | +++ | + |
| 9 | + | + |
| 10 | +++ | ++ |
| 11 | + | + |
| 12 | + | + |
| 13 | +++ | + |
| 14 | +++ | + |
| 15 | +++ | + |
| 16 | +++ | + |
| 17 | + | + |
| 18 | +++ | + |
| 22 | +++ | + |
| 24 | + | + |
| 25 | ++ | + |
| 26 | ++ | + |
| 27 | +++ | + |
| 28 | +++ | + |
| 29 | ++ | + |
| 30 | +++ | + |
| 31 | + | + |
| 32 | +++ | + |
| 33 | +++ | + |
| 34 | +++ | + |
| 39 | +++ | + |
| 40 | + | + |
| 41 | ++ | + |
| 42 | ++ | + |
| 43 | +++ | ++ |
| 44 | + | + |
| 45 | +++ | + |
| 47 | + | + |
| 48 | + | + |
| 49 | +++ | + |
| 50 | + | + |

From these results, the blocking activity of Nav 1.7 and/or 1.8 is proven, whose application is readily performed by those skilled in the art in pharmaceutical compositions, which may comprise one or more of the aforementioned Formula I compounds, kits, in addition to uses in the treatment of pain-related pathologies.

In particular, these results indicate the possibility of using Formula (I) compounds in the preparation of drugs for the treatment of conditions such as peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia, and postsurgical neuralgia.

It should be understood that the embodiments described above are merely illustrative and that several modifications can be made by a person skilled in the art without departing from the scope of this invention. Consequently, the present invention should not be regarded as being limited to the illustrative specifications described in this application.

1. A compound of Formula (I): 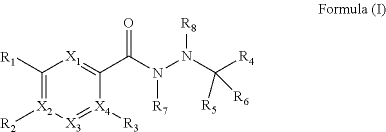
or a pharmaceutically acceptable salt, hydrate, solvate, ester and isomer thereof, wherein:
X₁, X₂, X₃, and X₄ are independently selected from the group consisting of carbon, CH, or nitrogen, wherein:
when at least one of the substituents X₂, X₄ is N or CH, the corresponding R (R₂, R₃) is null;
R₁ is: 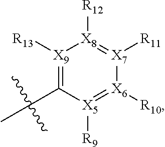
wherein:
X₅, X₆, X₇, X₈, and X₉ are independently selected from the group consisting of carbon, CH, or nitrogen, wherein:
when at least one of the substituents X₅-X₉ is N or CH, the corresponding R (R₉—R₁₃) is null;
R₉, R₁₀, R₁₁, R₁₂, and R₁₃ are independently selected from the group consisting of hydrogen, halogen, linear or branched C₁-C₆ alkyl, linear or branched C₁-C₆ alkoxy, C₃-C₇ cycloalkoxy, difluoromethyl, trifluoromethyl, linear or branched C₁-C₆ haloalkoxy;
R₂ and R₃ are independently selected from the group consisting of hydrogen, halogen, amine, hydroxy, and alkyl C₁-C₆ linear or branched, alkoxy C₁-C₆ linear or branched;
R₄ is: 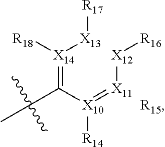
wherein:
X₁₀, X₁₁, X₁₂, X₁₃, and X₁₄ are independently selected from the group consisting of carbon, CH, or nitrogen, wherein:
when at least one of the substituents X₁₀-X₁₄ is N or CH, the corresponding R (R₁₄—R₁₈) is null,
R₁₄, R₁₅, R₁₆, R₁₇, and R₁₈ are independently selected from the group consisting of hydrogen, halogen, hydroxy(alkyl C₂-C₄ linear or branched), alkyl C₁-C₆ linear or branched, alkoxy C₁-C₆ linear or branched;
R₄ and R₈ are optionally substituted to form a piperidine, homopiperidine, homomorpholine, or pyrrolidine;
R₅ and R₆ are independently selected from the group consisting of hydrogen, linear or branched C₁-C₆ alkyl; and
R₇ and R₈ are independently selected from the group consisting of hydrogen, linear or branched C₁-C₆ alkyl.

2. The compound according to claim 1, selected from the group consisting of:
N′-(3,5-dimethoxybenzyl)-6-(4-methoxyphenyl)pyrazine-2-carbohydrazide (Compound 1);
N′-(4-chlorobenzyl)-6-(4-methoxyphenyl)pyrazine-2-carbohydrazide (Compound 2);
N′-(3,5-dimethoxybenzyl)-6-(4-isopropoxyphenyl)pyrazine-2-carbohydrazide (Compound 3);
N′-(4-chlorobenzyl)-6-(4-cyclopropoxyphenyl)pyrazine-2-carbohydrazide (Compound 4);
N′-(4-chlorobenzyl)-6-(4-chlorophenyl)pyrazine-2-carbohydrazide (Compound 5);
N-(6-chloro-3,4-dihydroisoquinoline-2(1H)-yl)-6-(4-chlorophenyl)pyrazine-2-carboxamide (Compound 6);
6-(4-(difluoromethoxy)phenyl)-N′-(3,5-dimethoxybenzyl)pyrazine-2-carbohydrazide (Compound 7);
6-(6-(difluoromethoxy)pyridin-3-yl)-N′-(3,5-dimethoxybenzyl)pyrazine-2-carbohydrazide (Compound 8);
N′-(4-chlorobenzyl)-6-(4-ethoxyphenyl)pyrazine-2-carbohydrazide (Compound 9);
N′-(3,5-dimethoxybenzyl)-6-(4-ethoxyphenyl)pyrazine-2-carbohydrazide (Compound 10);
6-(4-ethoxyphenyl)-N-(8-methoxy-1,3,4,5-tetrahydro-2H-benzo[c]azepine-2-yl)pyrazine-2-carboxamide (Compound 11);
6-(4-ethoxyphenyl)-N-(7-methoxy-2,3-dihydrobenzo[f][1,4]oxazepine-4(5H)-yl)pyrazine-2-carboxamide (Compound 12);
6-(4-ethoxyphenyl)-N-(5-methoxyisoindoline-2-yl)pyrazine-2-carboxamide (Compound 13);
6-(4-ethoxyphenyl)-N′-(5-methoxy-2-methylbenzyl)pyrazine-2-carbohydrazide (Compound 14);
6-(4-ethoxyphenyl)-N′-(2-fluoro-5-methoxybenzyl)pyrazine-2-carbohydrazide (Compound 15);
6-(4-ethoxyphenyl)-N′-(3-methoxybenzyl)pyrazine-2-carbohydrazide (Compound 16);
N′-(3,5-dimethoxybenzyl)-6-(4-ethoxyphenyl)-N′-methylpyrazine-2-carbohydrazide (Compound 17);
6-(4-ethoxyphenyl)-N′-((5-methoxy-2-methylpyridin-3-yl)methyl)pyrazine-2-carbohydrazide (Compound 18);
6-(4-ethoxyphenyl)-N′-(3-(1-hydroxyethyl)-5-methoxybenzyl)pyrazine-2-carbohydrazide (Compound 19);
6-(4-ethoxyphenyl)-N′-((2-fluoro-5-(1-hydroxyethyl)pyridin-3-yl)methyl)pyrazine-2-carbohydrazide (Compound 20);
6-(4-ethoxyphenyl)-N′-(2-fluoro-5-(1-hydroxyethyl)benzyl)pyrazine-2-carbohydrazide (Compound 21);
6-(4-ethoxyphenyl)-N′-(2-fluorobenzyl)pyrazine-2-carbohydrazide (Compound 22);
6-(4-ethoxyphenyl)-N′-(3-(1-hydroxyethyl)benzyl)pyrazine-2-carbohydrazide (Compound 23);
(S)—N′-(1-(3,5-dimethoxyphenyl)ethyl)-6-(4-ethoxyphenyl)pyrazine-2-carbohydrazide (Compound 24);
(R)—N′-(1-(3,5-dimethoxyphenyl)ethyl)-6-(4-ethoxyphenyl)pyrazine-2-carbohydrazide (Compound 25);
6-(4-ethoxyphenyl)-N′-((2-fluoro-5-methoxypyridin-3-yl)methyl)pyrazine-2-carbohydrazide (Compound 26);
N′-(3,5-dimethoxybenzyl)-6-(4-ethoxy-2-fluorophenyl)pyrazine-2-carbohydrazide (Compound 27);
N′-(3,5-dimethoxybenzyl)-6-(4-ethoxy-2-methylphenyl)pyrazine-2-carbohydrazide (Compound 28);
N′-(3,5-dimethoxybenzyl)-6-(6-ethoxypyridin-3-yl)pyrazine-2-carbohydrazide (Compound 29);
6-(6-ethoxypyridin-3-yl)-N′-(3-methoxybenzyl)pyrazine-2-carbohydrazide (Compound 30);
6-(6-ethoxypyridin-3-yl)-N′-((2-fluoro-5-methoxypyridin-3-yl)methyl)pyrazine-2-carbohydrazide (Compound 31);
6-(6-ethoxypyridin-3-yl)-N′-(2-fluoro-5-methoxybenzyl)pyrazine-2-carbohydrazide (Compound 32);
6-(6-ethoxypyridin-3-yl)-N′-(5-methoxy-2-methylbenzyl)pyrazine-2-carbohydrazide (Compound 33);
6-(6-ethoxypyridin-3-yl)-N′-((5-methoxy-2-methylpyridin-3-yl)methyl)pyrazine-2-carbohydrazide (Compound 34);
6-(6-ethoxypyridin-3-yl)-N′-(3-(1-hydroxyethyl)benzyl)pyrazine-2-carbohydrazide (Compound 35);
6-(6-ethoxypyridin-3-yl)-N′-(3-(1-hydroxyethyl)-5-methoxybenzyl)pyrazine-2-carbohydrazide (Compound 36);
6-(6-ethoxypyridin-3-yl)-N′-((2-fluoro-5-(1-hydroxyethyl)pyridin-3-yl)methyl)pyrazine-2-carbohydrazide (Compound 37);
6-(6-ethoxypyridin-3-yl)-N′-(2-fluoro-5-(1-hydroxyethyl)benzyl)pyrazine-2-carbohydrazide (Compound 38);
6-(6-ethoxypyridin-3-yl)-N′-(2-fluorobenzyl)pyrazine-2-carbohydrazide (Compound 39);
N′-(3,5-dimethoxybenzyl)-6-(6-ethoxy-4-methylpyridin-3-yl)pyrazine-2-carbohydrazide (Compound 40);
N′-(3,5-dimethoxybenzyl)-6-(6-ethoxy-2-methylpyridin-3-yl)pyrazine-2-carbohydrazide (Compound 41);
6-(6-cyclopropoxypyridin-3-yl)-N′-(3,5-dimethoxybenzyl)pyrazine-2-carbohydrazide (Compound 42);
N′-(3,5-dimethoxybenzyl)-6-(6-isopropoxypyridin-3-yl)pyrazine-2-carbohydrazide (Compound 43);
4-(4-chlorophenyl)-N-(3,5-dimethoxybenzyl)pyrimidine-2-carbohydrazide (Compound 44);
6-(4-ethoxyphenyl)-N-(7-methoxy-3,4-dihydroisoquinoline-2(1H)-yl)pyrazine-2-carboxamide (Compound 45);
6-(4-ethoxyphenyl)-N-(2-fluorobenzyl)pyridazine-4-carbohydrazide (Compound 46);
5-(4-ethoxyphenyl)-N-(2-fluorobenzyl)pyridazine-3-carbohydrazide (Compound 47);
4-(4-ethoxyphenyl)-N-(2-fluorobenzyl)picolinohydrazide (Compound 48);
5-(4-ethoxyphenyl)-N-(2-fluorobenzyl)nicotinohydrazide (Compound 49);
6-(4-ethoxyphenyl)-N-(2-fluorobenzyl)picolinohydrazide (Compound 50);
2-(4-ethoxyphenyl)-N-(2-fluorobenzyl)isonicotinohydrazide (Compound 51);
2-(4-ethoxyphenyl)-N-(2-fluorobenzyl)pyrimidine-4-carbohydrazide (Compound 52) and
4-(4-ethoxyphenyl)-N-(2-fluorobenzyl)pyrimidine-2-carbohydrazide (Compound 53).

3. The compound according to claim 1, characterized by being a blocker of voltage-gated sodium channels Nav 1.7 and/or Nav 1.8.

4. A pharmaceutical composition comprising a therapeutically effective amount of one or more compounds of Formula (I) or of a pharmaceutically acceptable salt, hydrate, solvate, and isomer thereof, as defined in claim 1, and one or more pharmaceutically acceptable excipients.

5. The pharmaceutical composition according to claim 4, characterized by being formulated as an oral, sublingual, nasal, parenteral, injectable, submuscular, topical, transdermal, ocular, or rectal composition.

6.-7. (canceled)

8. A method of treating, preventing, relieving, suppressing, and/or controlling pathologies related to neuropathic pain, characterized by the administration of an effective amount of at least one compound of Formula (I) or a pharmaceutically acceptable salt, hydrate, solvate and isomer thereof, as defined in claim 1.

9. The method according to claim 8, characterized by being for the treatment or prophylaxis of peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia, and postsurgical neuralgia.

10. The method according to claim 8, characterized by the administration of at least one compound of Formula (I) being selected from the group comprising oral, sublingual, nasal, parenteral, injectable, submuscular, topical, transdermal, ocular, and rectal routes.

11. A method of making the compound according to claim 1, comprising the steps:
(a) formation of the Formula IV or Formula VI intermediate: 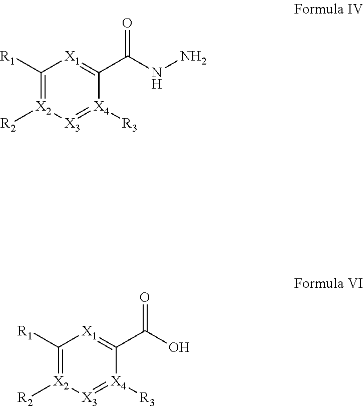
from the hydrazinolysis reaction or hydrolysis of a Formula V intermediate: 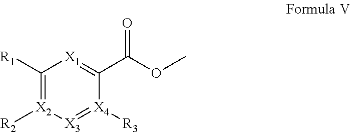
(b) formation of the Formula II intermediate: 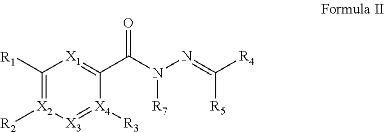
from condensation between Formula III intermediates: 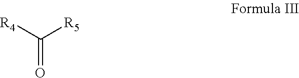
and Formula IV intermediates with or without the presence of a catalyst and a suitable solvent;
(c) Formula I compound is obtained: 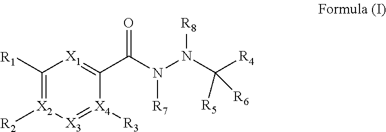
from the reduction of Formula II intermediates or from reactions and amide coupling between intermediates VI and VII with or without the presence of a catalyst and a suitable solvent: 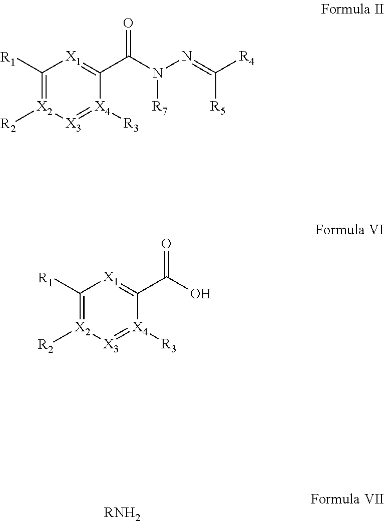
wherein:
X₁, X₂, X₃, and X₄ are independently selected from the group consisting of carbon, CH, or nitrogen, wherein:
when at least one of the substituents X₂, X₄ is N or CH, the corresponding R (R₂, R₃) is null;
R₁ is: 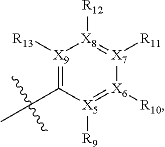
wherein:
X₅, X₆, X₇, X₈, and X₉ are independently selected from the group consisting of carbon, CH, or nitrogen, wherein:
when at least one of the substituents X₅-X₉ is N or CH, the corresponding R (R₉—R₁₃) is null;
R₉, R₁₀, R₁₁, R₁₂, and R₁₃ are independently selected from the group consisting of hydrogen, halogen, linear or branched C₁-C₆ alkyl, linear or branched C₁-C₆ alkoxy, C₃-C₇ cycloalkoxy, difluoromethyl, trifluoromethyl, linear or branched C₁-C₆ haloalkoxy;
R₂ and R₃ are independently selected from the group consisting of hydrogen, halogen, amine, hydroxy, and alkyl C₁-C₆ linear or branched, alkoxy C₁-C₆ linear or branched;
R₄ is: 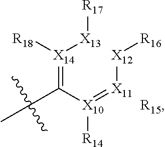
where:
X₁₀, X₁₁, X₁₂, X₁₃, and X₁₄ are independently selected from the group consisting of carbon, CH, or nitrogen, wherein:
when at least one of the substituents X₁₀-X₁₄ is N or CH, the corresponding R (R₁₄—R₁₈) is null,
R₁₄, R₁₅, R₁₆, R₁₇, and R₁₈ are independently selected from the group consisting of hydrogen, halogen, hydroxy(alkyl C₂-C₄ linear or branched), alkyl C₁-C₆ linear or branched, alkoxy C₁-C₆ linear or branched;
R₄ and R₈ are optionally substituted to form a piperidine, homopiperidine, homomorpholine, or pyrrolidine;
R₅ and R₆ are independently selected from the group consisting of hydrogen, linear or branched C₁-C₆ alkyl; and
R₇ and R₈ are independently selected from the group consisting of hydrogen, linear or branched C₁-C₆ alkyl.

12. The method according to claim 11, characterized by additionally comprising a step (d) of compound formation(s) of Formula (I) in which R₇ is methyl from the N-alkylation reaction of a compound obtained in step (c) with formaldehyde.

13. The method according to claim 11, characterized by the compound of Formula III of step (b) being an acetophenone derivative or a benzaldehyde derivative.

14. The method according to claim 12, characterized by in step (c) the step-reducing agent being selected between triethylsilane or sodium borohydride, and the acid being TFA.

15. A kit comprising a pharmaceutical composition as defined in claim 4 and an application device
